(19) United States
(12) Patent Application Publication
(10) Pub. No.: US 2015/0030630 A1
(43) Pub. Date: Jan. 29, 2015

(54) ADJUVANTED FORMULATIONS OF RABIES VIRUS IMMUNOGENS

(71) Applicant: Novartis AG, Basel (CH)

(72) Inventors: Siddhartha Jain, Troy, NY (US); Derek O'Hagan, Winchester, MA (US); Manmohan Singh, Cary, NC (US)

(73) Assignee: Novartis AG, Basel (CH)

(21) Appl. No.: 14/382,579

(22) PCT Filed: Mar. 7, 2013
(86) PCT No.: PCT/EP13/54547

§ 371 Date: Sep. 3, 2014

Related U.S. Application Data
(60) Provisional application No. 61/607,975, filed on Mar. 7, 2012.

(51) Int. Cl. A61K 39/39; A61K 39/205

(52) U.S. Cl. CPC A61K 39/39; A61K 39/205; A61K 2039/55511
USPC 424/224.1

(57) ABSTRACT

The efficacy of rabies vaccines can be enhanced by adjuvanting rabies virus immunogens with a mixture of a TLR agonist (preferably a TLR7 agonist) and an insoluble metal salt (preferably an aluminium salt). The TLR agonist is typically adsorbed to the metal salt. The rabies virus immunogen can also be adsorbed to the metal salt.

This application claims the benefit of U.S. Provisional Application No. 61/607,975, which was filed Mar. 7, 2012, the complete contents of which are hereby incorporated herein by reference for all purposes.

### TECHNICAL FIELD

The invention is in the field of rabies virus vaccines.

### BACKGROUND ART

The RABIPUR product, also known as RABAVERT, is a human vaccine against rabies virus [1]. The immunogen in this vaccine is an inactivated rabies virus. The virus is grown on purified chick embryo cells (PCEC) and is inactivated using β-propiolactone. Each dose includes at least 2.5 IU of inactivated Rabies virus (Flury LEP strain), in accordance with the World Health Organisation requirements, and is unadjuvanted. The vaccine is supplied as a lyophilised powder for reconstitution with sterile water (1 ml per dose) and subsequent injection, either intramuscularly or intradermally. It is administered (i) as a pre-exposure vaccine for people at risk of being infected, usually as 3 doses given over a 3-4 week period (ii) as a post-exposure vaccine, usually as 4-5 doses over a 3-4 week period or (iii) as a booster vaccine, initially 1 year after an earlier immunisation but thereafter every 2-5 years. Subjects may also receive anti-rabies immunoglobulin (e.g. 20 IU of human anti-rabies Ig), particularly in severe post-exposure vaccination settings.

Other human rabies vaccines include RABIVAC (prepared from viruses grown on human diploid cells), VAXIRAB (prepared using purified duck embryo cells), and various products prepared from viruses grown in vero cell culture (e.g. VERORAB [2], IMOVAX, CHENGDA). These are often used in similar ways to the RABIPUR product e.g. the VERORAB product is inactivated with β-propiolactone, formulated as a lyophilisate, and administered in unadjuvanted form, and its pre-exposure, post-exposure and boosting regimens are similar to those of RABIPUR.

Cattle vaccines have been adjuvanted with aluminium hydroxide, with or without avridine[3], and the RABISIN product for animal use, based on rabies virus glycoproteins, includes 1.7 mg of aluminium hydroxide per dose, but human rabies vaccines are typically unadjuvanted. The RVA human vaccine from BioPort Corporation was adsorbed to an aluminium salt adjuvant [4], but reference 5 asked whether human rabies vaccines should contain aluminium adjuvant and found that there is no advantage in its inclusion. Thus the authors recommended that aluminium-based adjuvants should be eliminated from rabies vaccines for human use.

### DISCLOSURE OF THE INVENTION

It is an object of the invention to provide further and improved rabies vaccines suitable for human use, and in particular to provide vaccines which overcome difficulties with existing vaccines.

One problem with existing human vaccines is that people do not realise that multiple doses will be required over a period of several weeks. For pre-exposure use, people often begin the vaccination regimen less than 4 weeks before the risk of exposure (e.g. less than 4 weeks before travelling abroad) and so the full regimen remains incomplete. For post-exposure use, people often do not complete the full regimen, particularly the 5-dose regimen. In these situations, therefore, patients do not receive the full benefit of the vaccines, and may not be protected against rabies disease.

A further problem with current vaccines is that they do not give long-lasting protection, hence the need for booster every 5 years or so.

Furthermore, reliable supply capacity for rabies vaccines is limited e.g. in late-2008 there was a severe shortage of supply in the USA.

Finally, existing vaccines have relatively high levels of residual antibiotics and, although these are safe, they are undesirable in terms of general product purity.

The inventors realise that all of these issues can be addressed by using a suitably adjuvanted vaccine. Some adjuvants are able to provide higher immune titers, and can achieve these more rapidly, thereby giving good protection with fewer doses on a shorter timescale. Similarly, adjuvants can provide long-lasting responses, thereby avoiding the need for frequent boosters. Finally, the use of adjuvants can give a vaccine which is equally immunogenic but includes less antigen, thus giving more individual doses from a fixed amount of virus, and also permitting the use of less antibiotic in a final vaccine.

To achieve these goals rabies vaccines can be adjuvanted with a mixture of a TLR agonist and an insoluble metal salt. The TLR agonist is typically adsorbed to the metal salt, as disclosed in reference 6, and the rabies virus antigen can also be adsorbed to the metal salt.

Therefore the invention provides an immunogenic composition comprising (i) an insoluble metal salt, (ii) a TLR agonist and (iii) a rabies virus immunogen. The composition preferably has one or more of the following characteristics:
The metal salt is an aluminium salt, such as an aluminium hydroxide.
The TLR agonist is a TLR7 agonist, and ideally an agonist of human TLR7.
The TLR agonist is adsorbed to the insoluble metal salt, ideally with at least 50% of the TLR agonist adsorbed.
The TLR agonist and the rabies immunogen are both adsorbed to the metal salt, ideally with at least 50% of the TLR agonist and at least 50% of the immunogen adsorbed.
The TLR agonist is a compound of formula lc herein, and ideally is compound ‘K2’ or a pharmaceutically acceptable salt thereof.
The composition is not lyophilized, and is not prepared by aqueous reconstitution of a lyophilizate.
The composition is formulated in aqueous form for distribution.
The composition does not include a disaccharide (e.g. sucrose), or has <20 mg/ml of disaccharide (e.g. sucrose).
The composition is free from polygeline (a cross-linked polymer of urea and polypeptides derived from degraded gelatin).
The composition is free from at least one of neomycin, chlortetracycline, and/or amphotericin B, and preferably is antibiotic-free.
The composition includes less than 2.5 IU per unit dose e.g. less than 5 IU/ml for a dosage volume of 0.5 ml, or less than 2.5 IU/ml for a dosage volume of 1 ml.
The composition is administered on a rapid dosing regimen, as discussed below.
The composition is administered in a dosage volume of between 0.05 and 0.9 ml e.g. a dosage volume of 0.1 ml or 0.5 ml.
The composition has an osmolality of between 200 mOsm/kg and 400 mOsm/kg,
The composition does not include human albumin.
The rabies virus immunogen is produced in Vero or MRC-5 cells.
   For example, the invention provides:
An immunogenic composition comprising (i) an insoluble metal salt (ii) a human TLR7 agonist and (iii) a rabies virus immunogen.
An immunogenic composition comprising (i) an insoluble aluminium salt (ii) a TLR agonist and (iii) a rabies virus immunogen.
An immunogenic composition comprising (i) an aluminium hydroxide adjuvant (ii) compound ‘K2’ or a pharmaceutically acceptable salt thereof, adsorbed to the aluminium hydroxide and (iii) an inactivated rabies virus.

The invention also provides an immunogenic composition comprising (i) an insoluble metal salt (ii) a TLR agonist and (iii) a rabies virus immunogen, wherein the composition has a pH between 6 and 8.

The invention also provides an immunogenic composition comprising (i) an insoluble metal salt (ii) a TLR agonist (iii) a rabies virus immunogen and (iv) a pH buffer e.g. with a pKa in the range of 5 to 9.

The invention also provides an immunogenic composition comprising (i) an insoluble metal salt (ii) a TLR agonist and (iii) a rabies virus immunogen, wherein the composition is antibiotic-free.

The invention also provides an immunogenic composition comprising (i) an insoluble metal salt (ii) a TLR agonist and (iii) a rabies virus immunogen at a concentration of less than 5 IU/ml e.g. at a concentration of less than 2.5 IU/ml.

The invention also provides an immunogenic composition in unit dose form, comprising (i) an insoluble metal salt (ii) a TLR agonist and (iii) a rabies virus immunogen, wherein the concentration of rabies virus immunogen is less than 2.5 IU per unit dose.

The invention also provides a process for preparing an immunogenic composition, wherein the process comprises mixing an insoluble metal salt, a TLR agonist, and a rabies virus immunogen. This process can provide the immunogenic compositions described above.

The invention also provides a process for preparing an immunogenic composition, comprising one of: (i) combining a rabies virus immunogen with a mixture comprising a TLR agonist and an insoluble metal salt; (ii) combining an insoluble metal salt with a mixture comprising a TLR agonist and a rabies virus immunogen; or (iii) combining a TLR agonist with a mixture comprising an insoluble metal salt and a rabies virus immunogen.

The invention also provides a composition comprising: (a) an adjuvant complex comprising a first TLR agonist adsorbed to an insoluble metal salt; (b) an adjuvant complex comprising a second TLR agonist adsorbed to an insoluble metal salt; and (c) a rabies virus immunogen.

The invention also provides a process for preparing an immunogenic composition comprising steps of (i) preparing an aqueous mixture of a TLR agonist and a soluble aluminium salt, and then adding a non-aluminium salt to the aqueous mixture in order to form a precipitated aluminium salt to which the TLR agonist is adsorbed; and (ii) mixing a rabies virus immunogen with the precipitated salt and its adsorbed agonist.

The invention also provides a process for preparing an immunogenic composition, comprising a step of mixing (i) an aqueous mixture of a TLR agonist and a soluble aluminium salt with (ii) a buffered aqueous mixture of a rabies virus immunogen, wherein the mixing step causes precipitation of an aluminium salt to which the TLR agonist and the immunogen are adsorbed. The invention also provides an immunogenic composition obtained or obtainable by this process.

The invention also provides a process for preparing a sterile immunogenic composition, comprising steps of combining (i) a rabies virus immunogen with (ii) a sterile complex of a TLR agonist and an insoluble metal salt. The sterile complex can be prepared by a process comprising steps of (a) mixing a TLR agonist and an insoluble metal salt such that the TLR agonist adsorbs to the insoluble metal salt to form the complex; and (b) sterilising the complex. Sterilisation can be conveniently achieved by autoclaving (or similar procedures [7]). As an alternative, the sterile complex can be prepared by (a) sterilising a solution or suspension of a TLR agonist and (b) combining the sterilised solution or suspension with a sterile insoluble metal salt; or by (a) sterilising an insoluble metal salt and (b) combining the sterilised insoluble metal salt with a sterile solution or suspension of a TLR agonist; or by combining (a) a sterile solution or suspension of a TLR agonist with (b) a sterile insoluble metal salt. Sterilisation of the TLR agonist solution/suspension can conveniently be achieved by sterile filtration, and this material can be prepared in concentrated form. Sterilisation of the insoluble metal salt can conveniently be achieved by autoclaving. The sterile insoluble metal salt will typically be an aqueous suspension.

The invention also provides a dosing regimen for administering an immunogenic composition of the invention, wherein the composition is administered: (a) to achieve pre-exposure protection by only 1 or 2 doses; (b) to achieve post-exposure protection by only 1, 2 or 3 does; or (c) by 3 separate doses within a week.

Insoluble Metal Salts

TLR agonists can adsorb to insoluble metal salts to form an adsorbed complex for adjuvanting rabies virus immunogens. For instance, they can be adsorbed to insoluble calcium salts (e.g. calcium phosphate) or, preferably, to insoluble aluminium salts. Such aluminium salts have a long history of use in vaccines.

Useful aluminium salts include, but are not limited to, aluminium hydroxide and aluminium phosphate adjuvants. Such salts are described e.g. in chapters 8 & 9 of reference 8. Aluminium salts which include hydroxide ions are the preferred insoluble metal salts for use with the present invention as these hydroxide ions can readily undergo ligand exchange. Thus preferred salts for adsorption of TLR agonists are aluminium hydroxide and/or aluminium hydroxyphosphate. These have surface hydroxyl moieties which can readily undergo ligand exchange with phosphorus-containing groups (e.g. phosphates, phosphonates) to provide stable adsorption. An aluminium hydroxide adjuvant is most preferred.

The adjuvants commonly known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ (chapter 9 of ref. 8). The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants e.g. with needle-like particles with diameters about 2 nm. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants commonly known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls (chapter 9 of reference 8).

The PO₄/Al³⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs, with primary particles in the range of 50 nm). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

In solution both aluminium phosphate and hydroxide adjuvants tend to form stable porous aggregates 1-10 μm in diameter [9].

A composition including a TLR agonist adsorbed to a metal salt can also include a buffer (e.g. a phosphate or a histidine or a Tris buffer). When such a composition includes a phosphate buffer, however, it is preferred that the concentration of phosphate ions in the buffer should be less than 50 mM e.g. <40 mM, <30 mM, <20 mM, <10 mM, or <5 mM, or between 1-15 mM. A histidine buffer is preferred e.g. between 1-50 mM, between 5-25 mM, or about 10 mM.

Because of the insolubility of adsorptive metal salts which are useful with the invention, compositions containing adsorbed TLR agonists will generally be suspensions having a cloudy appearance. This can mask contaminating bacterial growth and so a composition of the invention may include a preservative such as thiomersal or 2-phenoxyethanol. It is preferred that a composition should be substantially free from (e.g. <10 μg/ml) mercurial material e.g. thiomersal-free. Compositions containing no mercury are more preferred. Preservative-free compositions are also possible

A composition can include a mixture of both an aluminium hydroxide and an aluminium phosphate, and a TLR agonist may be adsorbed to one or both of these salts.

The concentration of Al⁺⁺⁺ in a composition for administration to a patient is preferably less than 10 mg/ml e.g. ≦5 mg/ml, ≦4 mg/ml, ≦3 mg/ml, ≦2 mg/ml, ≦1 mg/ml, etc. A preferred range of Al⁺⁺⁺ in a composition of the invention is between 0.3 and 1 mg/ml or between 0.3-0.5 mg/ml. A maximum of 0.85 mg/dose is preferred. Because the inclusion of a TLR agonist can improve the adjuvant effect of aluminium salts then the invention advantageously permits lower amounts of Al⁺⁺⁺ per dose, and so a composition of the invention can usefully include between 10 and 250 μg of Al⁺⁺⁺ per unit dose.

Current pediatric vaccines typically include at least 300 μg Al⁺⁺⁺. In concentration terms, a composition of the invention may have an Al⁺⁺⁺ concentration between 10 and 500 μg/ml e.g. between 10-300 μg/ml, between 10-200 μg/ml, or between 10-100 μg/ml.

In general, when a composition includes both a TLR agonist and an aluminium salt, the weight ratio of agonist to Al⁺⁺⁺ will be less than 5:1 e.g. less than 4:1, less than 3:1, less than 2:1, or less than 1:1. Thus, for example, with an Al⁺⁺⁺ concentration of 0.5 mg/ml the maximum concentration of TLR agonist would be 1.5 mg/ml. But higher or lower levels can be used.

It is preferred that at least 50% (by mass) of the agonist in the composition is adsorbed to the metal salt e.g. ≧60%, ≧70%, ≧80%, ≧85%, ≧90%, ≧92%, ≧94%, ≧95%, ≧96%, ≧97%, ≧98%, ≧99%, or even 100%.

TLR Agonists

Compositions of the invention include a TLR agonist i.e. a compound which can agonise a Toll-like receptor. Most preferably, a TLR agonist is an agonist of a human TLR. The TLR agonist can activate any of TLR1, TLR2, TLR3, TLR4, TLR5, TLR6, TLR7, TLR8, TLR9 or TLR11; preferably it can activate human TLR7.

Agonist activity of a compound against any particular Toll-like receptor can be determined by standard assays. Companies such as Imgenex and Invivogen supply cell lines which are stably co-transfected with human TLR genes and NFκB, plus suitable reporter genes, for measuring TLR activation pathways. They are designed for sensitivity, broad working range dynamics and can be used for high-throughput screening. Constitutive expression of one or two specific TLRs is typical in such cell lines. See also reference 10. Many TLR agonists are known in the art e.g. reference 11 describes certain lipopeptide molecules that are TLR2 agonists, references 12 to 15 each describe classes of small molecule agonists of TLR7, and references 16 & 17 describe TLR7 and TLR8 agonists for treatment of diseases.

A TLR agonist used with the invention ideally includes at least one adsorptive moiety. The inclusion of such moieties in TLR agonists allows them to adsorb to insoluble metal salts (e.g. by ligand exchange or any other suitable mechanism) and improves their immunological behaviour (see reference 6). Phosphorus-containing adsorptive moieties are particularly useful, and so an adsorptive moiety may comprise a phosphate, a phosphonate, a phosphinate, a phosphonite, a phosphinite, etc.

Preferably the TLR agonist includes at least one phosphonate group.

Thus, in preferred embodiments, a composition of the invention includes a TLR7 agonist which includes a phosphonate group. This phosphonate group can allow adsorption of the agonist to an insoluble metal salt, such as to an aluminium salt.

TLR agonists useful with the invention may include a single adsorptive moiety, or may include more than one e.g. between 2 and 15 adsorptive moieties. Typically a compound will include 1, 2 or 3 adsorptive moieties.

Phosphorus-containing TLR agonists useful with the invention can be represented by formula (Al):

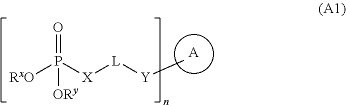

wherein:
R^{X} and R^{Y} are independently selected from H and C₁-C₆ alkyl;
X is selected from a covalent bond, 0 and NH;
Y is selected from a covalent bond, 0, C(O), S and NH;
L is a linker e.g. selected from, C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
each p is independently selected from 1, 2, 3, 4, 5 and 6;
q is selected from 1, 2, 3 and 4;
n is selected from 1, 2 and 3; and
A is a TLR agonist moiety.

In one embodiment, the TLR agonist according to formula (Al) is as follows: R^{X} and R^{Y} are H; X is O; L is selected from C₁-C₆ alkylene and —((CH₂)ₚO)_{q}(CH₂)ₚ— each optionally substituted with 1 to 2 halogen atoms; p is selected from 1, 2 and 3; q is selected from 1 and 2; and n is 1. Thus in these embodiments the adsorptive moiety comprises a phosphate group.

In other embodiments, the TLR agonist according to formula (A1) is as follows: R^{X} and R^{Y} are H; X is a covalent bond; L is selected from C₁-C₆ alkylene and —((CH₂)ₚO)_{q}(CH₂)ₚ— each optionally substituted with 1 to 2 halogen atoms; p is selected from 1, 2 or 3; q is selected from 1 or 2; and n is 1. Thus in these embodiments the adsorptive moiety comprises a phosphonate group.

Useful ‘A’ moieties for formula (Al) include, but are not limited to, radicals of any of the following compounds, defined herein or as disclosed in references 4-17 and 29-47:

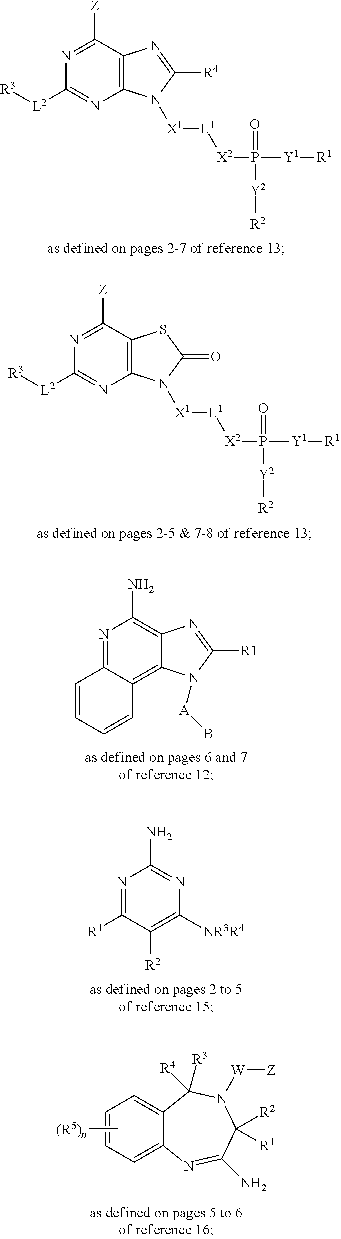

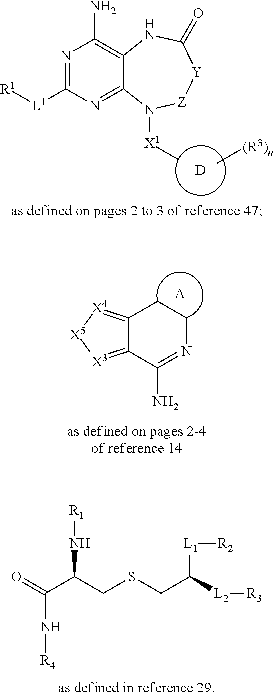

In some embodiments, the TLR agonist moiety ‘A’ has a molecular weight of less than 1000 Da. In some embodiments, the TLR agonist of formula (Al) has a molecular weight of less than 1000 Da.

Preferred TLR agonists are water-soluble. Thus they can form a homogenous solution when mixed in an aqueous buffer with water at pH 7 at 25° C. and 1 atmosphere pressure to give a solution which has a concentration of at least 50 μg/ml. The term “water-soluble” thus excludes substances that are only sparingly soluble under these conditions.

Useful TLR agonists include those having formula (C), (D), (E), (F), (G), (H), (I), (II), (J) or (K) as described in more detail below. Other useful TLR agonists are compounds 1 to 102 as defined in reference 6. Preferred TLR7 agonists have formula (K), such as compound K2 identified below. These can be used as salts e.g. the arginine salt of K2.

Preferred TLR4 agonists are analogs of monophosphoryl lipid A (MPL). For instance, a useful TLR4 agonist is a 3d-MPL (i.e. 3-O-deacylated monophosphoryl lipid A; also known as 3-de-O-acylated monophosphoryl lipid A or 3-O-desacyl-4′-monophosphoryl lipid A). The name indicates that position 3 of the reducing end glucosamine in monophosphoryl lipid A is de-acylated. It has been prepared from a heptoseless mutant of Salmonella minnesota, and is chemically similar to lipid A but lacks an acid-labile phosphoryl group and a base-labile acyl group. It activates cells of the monocyte/macrophage lineage and stimulates release of several cytokines, including IL-1, IL-12, TNF-α and GM-CSF. Preparation of 3d-MPL was originally described in reference 18, and the product has been manufactured and sold by Corixa Corporation. It is present in the AS04 adjuvant used by GlaxoSmithKline. Further details can be found in references 19 to 22. In some embodiments, however, the invention does not use a combination of aluminium phosphate and 3dMPL.

Typical compositions include 3d-MPL at a concentration of between 25 μg/ml and 200 μg/ml e.g. in the range 50-150 μg/ml, 75-125 μg/ml, 90-110 μg/ml, or about 100 μg/ml. It is usual to administer between 25-75 μg of 3d-MPL per dose e.g. between 45-55 μg, or about 50 μg 3d-MPL per dose.

3d-MPL can take the form of a mixture of related molecules, varying by their acylation (e.g. having 3, 4, 5 or 6 acyl chains, which may be of different lengths). The two glucosamine (also known as 2-deoxy-2-amino-glucose) monosaccharides are N-acylated at their 2-position carbons (i.e. at positions 2 and 2′), and there is also O-acylation at the 3′ position. The group attached to carbon 2 has formula —NH—CO—CH₂—CR¹R^{1′}. The group attached to carbon 2′ has formula —NH—CO—CH₂—CR²R^{2′}. The group attached to carbon 3′ has formula —O—CO—CH₂—CR³R^{3′}. A representative structure is:

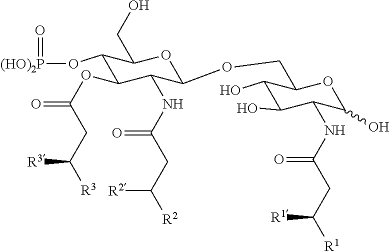

Groups R′, R² and R³ are each independently (CH₂)CH₃. The value of n is preferably between 8 and 16, more preferably between 9 and 12, and is most preferably 10.

Groups R^{1′}, R^{2′} and R^{3′} can each independently be: (a) —H; (b) —OH; or (c) —O—CO—R⁴, where R⁴ is either —H or —(CH₂)ₘ—CH₃, wherein the value of m is preferably between 8 and 16, and is more preferably 10, 12 or 14. At the 2 position, m is preferably 14. At the 2′ position, m is preferably 10. At the 3′ position, m is preferably 12. Groups R^{1′}, R^{2′} and R^{3′} are thus preferably —O-acyl groups from dodecanoic acid, tetradecanoic acid or hexadecanoic acid.

When all of R^{1′}, R^{2′} and R^{3′} are H then the 3d-MPL has only 3 acyl chains (one on each of positions 2, 2′ and 3′). When only two of R^{1′}, R^{2′} and R^{3′} are H then the 3d-MPL can have 4 acyl chains. When only one of R^{1′}, R^{2′} and R^{3′} is H then the 3d-MPL can have 5 acyl chains. When none of R^{1′}, R^{2′} and R^{3′} is H then the 3d-MPL can have 6 acyl chains. The 3d-MPL used according to the invention can be a mixture of these forms, with from 3 to 6 acyl chains, but it is preferred to include 3d-MPL with 6 acyl chains in the mixture, and in particular to ensure that the 6 acyl chain form makes up at least 10% by weight of the total 3d-MPL e.g. ≧20%, ≧30%, ≧40%, ≧50% or more. 3d-MPL with 6 acyl chains has been found to be the most adjuvant-active form.

Thus the most preferred form of 3d-MPL for use with the invention is:

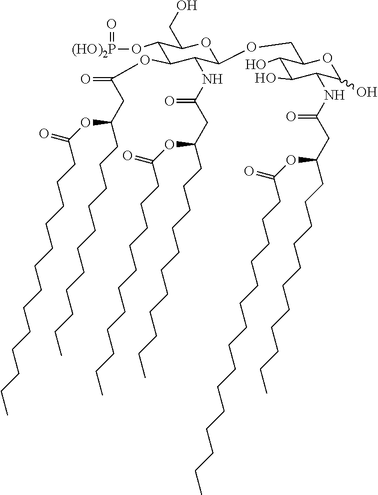

Where 3d-MPL is used in the form of a mixture then references to amounts or concentrations of 3d-MPL in compositions of the invention refer to the combined 3d-MPL species in the mixture.

In aqueous conditions, 3d-MPL can form micellar aggregates or particles with different sizes e.g. with a diameter <150 nm or >500 nm. Either or both of these can be used with the invention, and the better particles can be selected by routine assay. Smaller particles (e.g. small enough to give a clear aqueous suspension of 3d-MPL) are preferred for use according to the invention because of their superior activity [23]. Preferred particles have a mean diameter less than 150 nm, more preferably less than 120 nm, and can even have a mean diameter less than 100 nm. In most cases, however, the mean diameter will not be lower than 50 nm. Where 3d-MPL is adsorbed to aluminum phosphate then it may not be possible to measure the 3D-MPL particle size directly, but particle size can be measured before adsorption takes place. Particle diameter can be assessed by the routine technique of dynamic light scattering, which reveals a mean particle diameter. Where a particle is said to have a diameter of x nm, there will generally be a distribution of particles about this mean, but at least 50% by number (e.g. ≧60%, ≧70%, ≧80%, ≧90%, or more) of the particles will have a diameter within the range x±25%.

A composition of the invention can include more than one TLR agonist. These two agonists are different from each other and they can target the same TLR or different TLRs. Both agonists can be adsorbed to a metal salt.

In some embodiments, the invention does not encompass compositions which include avridine.

Rabies Virus Immunogens

Compositions of the invention include a rabies virus immunogen e.g. as described in chapter 27 of reference 24. The immunogen will generally be an inactivated rabies virus virion. These immunogens can be prepared by, in basic terms, inactivation of cell-free virus, such as the supernatant (usually concentrated and/or purified e.g. using density gradient centrifugation) of a cell culture followed by concentration of the inactivated virus. Inactivation is typically by β-propiolactone treatment (e.g. at a 1:4000 ratio), although formalin and ultraviolet light treatment are also in use. Concentration of culture supernatants can be achieved by ultrafiltration, zonal centrifugation, ultracentrifugation, etc. Concentration of inactivated virus can be by ultrafiltration.

The vaccine strain can be grown on any suitable cellular substrate, such as chicken embryo (PCEC), duck embryo, human cultured fibroblasts, WI-38, MRC-5, fetal rhesus lung, primary Syrian hamster kidney, the vero cell line, the MDCK cell line, etc. (see chapters 22-33 of reference 27). Viruses grown on these different cell substrates can be distinguished on the basis of their glycoforms e.g. viruses grown in vero cells have simian glycoforms, whereas PCEC and duck cells produce avian glycoforms, and MRC-5 cells produce human glycoforms. Older methods, which grew virus on nerve tissue (e.g. rabbit spinal cord, or brain from rabbit, sheep, goat or suckling mouse; (see chapters 19-21 of reference 27), are not preferred because of the risk of contamination.

The rabies virus immunogen can be prepared from any suitable rabies virus strain. Known strains of rabies virus include, but are not limited to: AVO1, CVS, ERA, Kelev, HEP-FLURY, Nishigahara RCEH, Ontario fox, Ontario skunk, Pasteur/PV, Pittman Moore (PM), Street Alabama Dufferin (SAD) B19, SAD-Bern, ERA, SAG, SAG2, Vnukovo-32, Eth2003, strain RC-HL, Nishigahara, SHBRV-18, SRV9, Ni—CE, Flury-LEP, and Kissling rabies virus strain. Strains of particular importance for vaccine production are PV, Pitman-Moore, CVS, Flury LEP, Flury HEP, Kelev, and ERA. Two strains which are particularly useful are the Pitman-Moore Strain (or the Wistar rabies PM/WI38-1503-3M strain) as used in VEROLAB, and the Flury LEP strain as used in RABIPUR.

An immunogenic composition of the invention will include an effective dose of the rabies virus immunogen. For human intramuscular injection a unit dose should, according to WHO guidelines, include ≧2.5 IU of rabies virus immunogen. This potency can be measured by standard protocols e.g. see references 25 (Annex 2), 26 (Annex 1) & 27 (chapters 36-43). Thus a vaccine of the invention can include ≧2.5 IU of rabies virus immunogen per unit dose. In advantageous embodiments of the invention, however, protection can be achieved by using a lower amount of immunogen i.e. less than 2.5 IU per unit dose [28]. Thus a vaccine can include 0.1-2.4 IU/dose, such as 0.5-2.0 IU/dose, or between 1.0-2.0 or 1.0-1.5 or 1.5-2.0 IU/dose. With a dosage volume of 1 ml the concentration of immunogen in IU/ml will be the same as the IU/dose; with a smaller dosage volume, such as 0.5 ml, the concentration will change accordingly e.g. a composition can include rabies immunogen at a concentration of ≧5 IU/ml, or between 0.2-4.8 IU/ml, etc.

In some embodiments, a composition of the invention includes a rabies virus immunogen and also an immunogen from a different organism (e.g. from a bacterium or from another virus).

Formulae (C), (D), (E) and (H) TLR 7 Agonists

The TLR agonist can be a compound according to any of formulae (C), (D), (E), and (H):

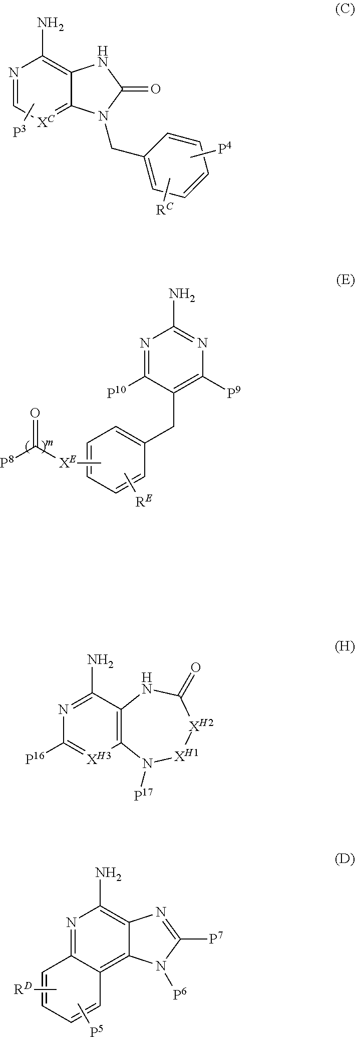

wherein:
(a) P³ is selected from H, C₁-C₆alkyl, CF₃, and —((CH₂)ₚO)_{q}(CH₂)ₚOₛ— and —Y-L-X—P(O)(OR^{X})(OR^{Y}); and P⁴ is selected from H, C₁-C₆alkyl, —C₁-C₆alkylaryl and —Y-L-X—P(O)(OR^{X})(OR^{Y}); with the proviso that at least one of P³ and P⁴ is —Y-L-X—P(O)(OR^{X})(OR^{Y}),
(b) P⁵ is selected from H, C₁-C₆alkyl, and —Y-L-X—P(O)(OR^{X})(OR^{Y}); P⁶ is selected from H, C₁-C₆alkyl each optionally substituted with 1 to 3 substituents selected from C₁-C₄alkyl and OH, and —Y-L-X—P(O)(OR^{X})(OR^{Y}); and P⁷ is selected from H, C₁-C₆alkyl, —((CH₂)ₚO)₄CH₂)ₚOₛ—, —NHC₁-C₆alkyl and —Y-L-X—P(O)(OR^{X})(OR^{Y}); with the proviso that at least one of P⁵, P⁶ and P⁷ is —Y-L-X—P(O)(OR^{X})(OR^{Y});
(c) P⁸ is selected from H, C₁-C₆alkyl, C₁-C₆alkoxy, —NHC₁-C₆alkyl each optionally substituted with OH, and —Y-L-X—P(O)(OR^{X})(OR^{Y}); and P⁹ and P¹⁹ are each independently selected from H, C₁-C₆alkyl, C₁-C₆alkoxy, —NHC₁-C₆alkyl each optionally substituted with OH and C₁-C₆alkyl, and —Y-L-X—P(O)(OR^{X})(OR^{Y}); with the proviso that at least one of P⁸, P⁹ or P¹⁰ is —Y-L-X—P(O)(OR^{X})(OR^{Y});
(d) P¹⁶ and each P¹⁸ are each independently selected from H, C₁-C₆alkyl, and —Y-L-X—P(O)(OR^{X})(OR^{Y}); P¹⁷ is selected from H, C₁-C₆alkyl, aryl, heteroaryl, C₁-C₆alkylaryl, C₁-C₆alkyl heteroaryl, C₁-C₆alkylaryl-Y-L-X—P(O)(OR^{X})(OR^{Y}) and —Y-L-X—P(O)(OR^{X})(OR^{Y}), each optionally substituted with 1 to 2 substituents selected from C₁-C₆alkyl or heterocyclyl with the proviso that at least one of P¹⁶, P¹⁷ or a P¹⁸ contains a —Y-L-X—P(O)(OR^{X})(OR^{Y}) moiety;
R^{X} and R^{Y} are independently selected from H and C₁-C₆alkyl;
R^{C}, R^{D} and R^{H} are each independently selected from H and C₁-C₆alkyl;
X^{C} is selected from CH and N;
R^{E} is selected from H, C₁-C₆alkyl, C₁-C₆alkoxy, C(O)C₁-C₆alkyl, halogen and —((CH₂)ₚO)_{q}(CH₂)ₚ—;
X^{E} is selected from a covalent bond, CR^{E2}R^{E3} and NR^{E4};
R^{E2}, R^{E3} and R^{E4} are independently selected from H and C₁-C₆alkyl;
X^{H1}-X^{H2} is selected from —CR^{H2}R^{H3}—, —CR^{H2}R^{H3}—CR^{H2}R^{H3}—, —C(O)CR^{H2}R^{H3}—, —C(O)CR^{H2}R^{H3}—, —CR^{H2}R^{H3}C(O)—, —NR^{H4}C(O)—, C(O)NR^{H4}—, CR^{H2}R^{H3}S(O)₂ and CR^{H2}═CR^{H2}—;
R^{H2}, R^{H3} and R^{H4} are each independently selected from H, C₁-C₆alkyl and P¹⁸;
X^{H3} is selected from N and CN;
X is selected from a covalent bond, 0 and NH;
Y is selected from a covalent bond, 0, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂;
m is selected from 0 or 1;
each p is independently selected from 1, 2, 3, 4, 5 and 6;
q is selected from 1, 2, 3 and 4; and
s is selected from 0 and 1.

Formula (G)—TLR8 Agonist

The TLR agonist can be a compound according to formula (G):

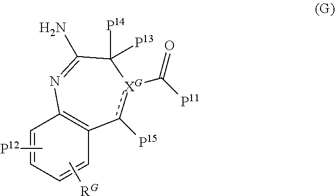

wherein:
P¹¹ is selected from H, C₁-C₆alkyl, C₁-C₆ alkoxy, NR^{V}R^{W} and —Y-L-X—P(O)(OR^{X})(OR^{Y});
P¹² is selected from H, C₁-C₆alkyl, aryl optionally substituted by C(O)NR^{V}R^{W}, and —Y-L-X—P(O)(OR^{X})(OR^{Y});
P¹³, P¹⁴ and P¹⁵ are independently selected from H, C₁-C₆alkyl, C₁-C₆ alkoxy and —Y-L-X—P(O)(OR^{X})(OR^{Y});
with the proviso that at least one of P¹¹, P¹², P¹³, P¹⁴ or P¹⁵ is Y-L-X—P(O)(OR^{X})(OR^{Y});
R^{V} and R^{W} are independently selected from H, C₁-C₆alkyl or together with the nitrogen atom to which they are attached form a 4 to 7 remembered heterocyclic ring;
X^{G} is selected from C, CH and N;
[symbol] represents an optional double bond, wherein X^{G} is C if [symbol] is a double bond; and
R^{G} is selected from H and C₁-C₆alkyl;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂;
each p is independently selected from 1, 2, 3, 4, 5 and 6 and
q is selected from 1, 2, 3 and 4.

Formulae (I) and (II)—TLR7 Agonists [13]

The TLR agonist can be a compound according to formula (I) or formula (II):

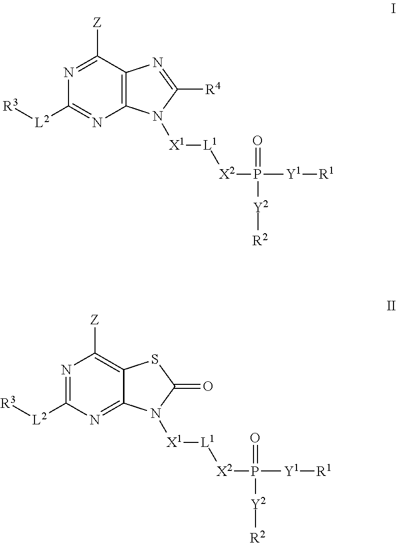

wherein:
Z is —NH₂ or —OH;
X¹ is alkylene, substituted alkylene, alkenylene, substituted alkenylene, alkynylene, substituted alkynylene, carbocyclylene, substituted carbocyclylene, heterocyclylene, or substituted heterocyclylene;
L¹ is a covalent bond, arylene, substituted arylene, heterocyclylene, substituted heterocyclylene, carbocyclylene, substituted carbocyclylene, —S—, —S(O)—, S(O)₂, —NR⁵—, or —O—
X² is a covalent bond, alkylene, or substituted alkylene;
L² is NR⁵—, —N(R⁵)C(O)—, —O—, —S—, —S(O)—, S(O)₂, or a covalent bond;
R³ is H, alkyl, substituted alkyl, heteroalkyl, substituted heteroalkyl, alkenyl, substituted alkenyl, aryl, substituted aryl, arylalkyl, substituted arylalkyl, heterocyclyl, substituted heterocyclyl, heterocyclylalkyl, or substituted heterocyclylalkyl;
Y¹ and Y² are each independently a covalent bond, —O— or —NR⁵—; or —Y′R′ and —Y²—R² are each independently —O—N═C(R⁶R²);
R¹ and R² are each independently H, alkyl, substituted alkyl, carbocyclyl, substituted carbocyclyl, heterocyclyl, substituted heterocyclyl, alkenyl, substituted alkenyl, alkynyl, substituted alkynyl, arylalkyl, substituted arylalkyl, heterocyclylalkyl, substituted heterocyclylalkyl, -alkylene-C(O)—O—R⁵, -(substituted alkylene)-C(O)—O—R⁵, -alkylene-O—C(O)—R⁵, -(substituted alkylene)-O—C(O)—R⁵, -alkylene-O—C(O)—O—R⁵, or -(substituted alkylene)-O—C(O)—O—R⁵
R⁴ is H, halogen, —OH, —O-alkyl, —O-alkylene-O—C(O)—O—R⁵, —O—C(O)—O—R⁵, —SH, or —NH(R⁵);
each R⁵, R⁶, and R⁷ are independently H, alkyl, substituted alkyl, carbocyclyl, substituted carbocyclyl, heterocyclyl, substituted heterocyclyl, alkenyl, substituted alkenyl, alkynyl, substituted alkynyl, arylalkyl, substituted arylalkyl, heterocyclylalkyl, or substituted heterocyclylalkyl.

Formula (J)—TLR2 agonists [29]

The TLR agonist can be a compound according to formula (J):

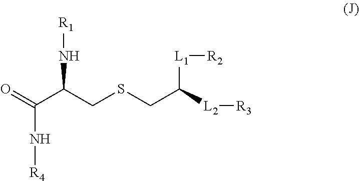

wherein:

R¹ is H, —C(O)—C₇-C₁₈alkyl or C(O)— C₁-C₆alkyl;

R² is C₇-C₁₈alkyl;

R³ is C₇-C₁₈alkyl;

L₁ is —CH₂OC(O)—, —CH₂O—, —CH₂NR⁷C(O)— or —CH₂OC(O)NR⁷—;

L₂ is —OC(O)—, —O—, —NR⁷C(O)— or —OC(O)NR⁷—;

R⁴ is -L₃R⁵ or -L₄R⁵;

R⁵ is N(R⁷)₂, —OR⁷, —P(O)(OR⁷)₂, —C(O)OR⁷, —NR⁷C(O)L₃R⁸, —NR⁷C(O)L₄R⁸, —OL₃R⁶, —C(O)NR⁷L₃R⁸, —C(O)NR⁷L₄R⁸, —S(O)₂OR⁷, —OS(O)₂OR⁷, C₁-C₆alkyl, a C₆aryl, a C₁₀aryl, a C₁₀aryl, 5 to 14 ring membered heteroaryl containing 1 to 3 heteroatoms selected from O, S and N, C₃-C₈cycloalkyl or a 5 to 6 ring membered heterocycloalkyl containing 1 to 3 heteroatoms selected from O, S and N, wherein the aryl, heteroaryl, cycloalkyl and heterocycloalkyl of R⁵ are each unsubstituted or the aryl, heteroaryl, cycloalkyl and heterocycloalkyl of R⁵ are each substituted with 1 to 3 substituents independently selected from —OR⁹, -OL₃R⁶, -OL₄R⁶, —OR⁷, and —C(O)OR⁷;

L₃ is a C₁-C₁₀alkylene, wherein the C₁-C₁₀alkylene of L₃ is unsubstituted, or the C₁-C₁₀alkylene of L₃ is substituted with 1 to 4 R⁶ groups, or the C₁-C₁₀alkylene of L₃ is substituted with 2 C₁-C₆alkyl groups on the same carbon atom which together, along with the carbon atom they are attached to, form a C₃-C₈cycloakyl;

L₄ is —((CR⁷R⁷)ₚO)_{q}(CR¹⁰R¹⁰)ₚ— or —(CR¹¹R¹¹), wherein each R¹¹ is a C₁-C₆alkyl groups which together, along with the carbon atom they are attached to, form a C₃-C₈cycloakyl;

each R⁶ is independently selected from halo, C₁-C₆alkyl, C₁-C₆alkyl substituted with 1-2 hydroxyl groups, —OR⁷, —N(R⁷)₂, —C(O)OH, —C(O)N(R⁷)₂, —P(O)(OR⁷)₂, a C₆aryl, a C₁₀aryl and a C₁₄aryl;

each R⁷ is independently selected from H and C₁-C₆alkyl;

R⁸ is selected from —SR′, —C(O)OH, —P(O)(OR⁷)₂, and a 5 to 6 ring membered heterocycloalkyl containing 1 to 3 heteroatoms selected from O and N;

R⁹ is phenyl;

each R¹⁹ is independently selected from H and halo;

each p is independently selected from 1, 2, 3, 4, 5 and 6, and

q is 1, 2, 3 or 4.

Preferably R⁵ is P(O)(OR⁷)₂, —NR⁷C(O)L₃-P(O)(OR⁷)₂, —NR⁷C(O)L₄-P(O)(OR⁷)₂, —OL₃-P(O)(OR⁷)₂, —C(O)NR⁷L₃-P(O)(OR⁷)₂, or —C(O)NR⁷L₄-P(O)(OR⁷)₂.

In some embodiments of (J), R₁ is H. In other embodiments of (J), R₁ is —C(O)—C₁₅alkyl;

In some embodiments of (J): (i) L₁ is —CH₂OC(O)— and L₂ is —OC(O)—, —O—, —NR⁷C(O)— or —OC(O)NR⁷—; or (ii) or L₁ is —CH₂O— and L₂ is —OC(O)—, —O—, —NR⁷C(O)— or —OC(O)NR⁷—; or (iii) L₁ is —CH₂NR⁷C(O)— and L₂ is —OC(O)—, —O—, —NR⁷C(O)— or —OC(O)NR⁷—; or (iv) L₁ is —CH₂OC(O)NR⁷— and L₂ is —OC(O)—, —O—, NR⁷C(O)— or —OC(O)NR⁷—.

In some embodiments of (J): (i) L₁ is —CH₂OC(O)— and L₂ is —OC(O)—; or (ii) L₁ is —CH₂O— and L₂ is —O—; or (iii) L₁ is —CH₂O— and L₂ is —NHC(O)—; or (iv) L₁ is —CH₂OC(O)NH— and L₂ is —OC(O)NH—.

In some embodiments of (J), (i) R² is —C₁₁alkyl and R³ is —C₁₁alkyl; or (ii) R² is —C₁₀alkyl and R³ is —C₁₀alkyl; or (iii) R² is —C₁₀alkyl and R³ is —C₁₁alkyl; or (iv) R² is —C₁₂alkyl and R³ is —C₁₂alkyl; or (v) R² is —C₇alkyl and R³ is —C₇alkyl; or (vi) R² is —C₉alkyl and R³ is —C₉alkyl; or (vii) R² is —C₈alkyl and R³ is —C₈alkyl; or (viii) R² is —C₁₃alkyl and R³ is —C₁₃alkyl; or (ix) R² is —C₁₂alkyl and R³ is —C₁₁alkyl; or (x) R² is —C₁₂alkyl and R³ is —C₁₂alkyl; or (xi) R² is —C₁₀alkyl and R³ is —C₁₀alkyl; or (xii) R² is —C₁₅alkyl and R³ is —C₁₅alkyl.

In some embodiments of (J), R² is —C₁₁alkyl and R³ is —C₁₁alkyl.

In some embodiments of (J), L₃ is a C₁-C₁₀alkylene, wherein the C₁-C₁₀alkylene of L₃ is unsubstituted or is substituted with 1 to 4 R⁶ groups.

In some embodiments of (J): L₄ is —((CR⁷R⁷)ₚO)_{q}(CR¹⁰R¹⁰)ₚ; each R¹⁰ is independently selected from H and F; and each p is independently selected from 2, 3, and 4.

In some embodiments of (J), each R⁶ is independently selected from methyl, ethyl, i-propyl, i-butyl, —CH₂OH, —OH, —F, —NH₂, —C(O)OH, —C(O)NH₂, —P(O)(OH)₂ and phenyl.

In some embodiments of (J), each R⁷ is independently selected from H, methyl and ethyl.

Formula (K) [30]

The TLR agonist can be a compound according to formula (K):

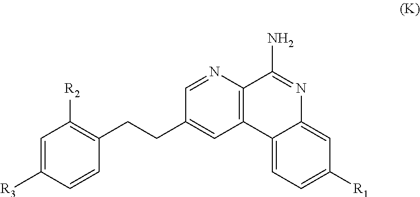

wherein:
R¹ is H, C₁-C₆alkyl, —C(R⁵)₂OH, -L¹R⁵, -L¹R⁶, -L²R⁵, -L²R⁶, —OL²R⁵, or —OL²R⁶;
L¹ is —C(O)— or —O—;
L² is C₁-C₆alkylene, C₂-C₆alkenylene, arylene, heteroarylene or —((CR⁴R⁴)ₚO)_{q}(CH₂)ₚ—, wherein the C₁-C₆alkylene and C₂-C₆alkenylene of L² are optionally substituted with 1 to 4 fluoro groups;

each L³ is independently selected from C₁-C₆alkylene and —((CR⁴R⁴)ₚO)_{q}(CH₂)ₚ—, wherein the C₁-C₆alkylene of L³ is optionally substituted with 1 to 4 fluoro groups;

L⁴ is arylene or heteroarylene;

R² is H or C₁-C₆alkyl;

R³ is selected from C₁-C₄alkyl, -L³R⁵, -L¹R⁵, -L³R⁷, -L³L⁴L³R⁷, -L³L⁴R⁵, -L³L⁴L³R⁵, —OL³R⁵, —OL³R⁷, —OL³L⁴R⁷, —OL³L⁴L³R⁷, —OR⁸, —OL³L⁴R⁵, —OL³L⁴L³R⁵ and —C(R⁵)₂OH;
each R⁴ is independently selected from H and fluoro;
R⁵ is —P(O)(OR⁹)₂,
R⁶ is —CF₂P(O)(OR⁹)₂ or —C(O)OR¹⁹;
R⁷ is —CF₂P(O)(OR⁹)₂ or —C(O)OR¹⁹;
R⁸ is H or C₁-C₄alkyl;
each R⁹ is independently selected from H and C₁-C₆alkyl;
R¹⁹ is H or C₁-C₄alkyl;
each p is independently selected from 1, 2, 3, 4, 5 and 6, and
q is 1, 2, 3 or 4.

The compound of formula (K) is preferably of formula (K′):

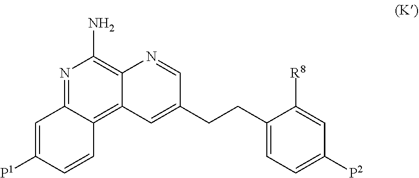

wherein:
P¹ is selected from H, C₁-C₆alkyl optionally substituted with COOH and —Y-L-X—P(O)(OR^{X})(OR^{Y});
P² is selected from H, C₁-C₆alkyl, C₁-C₆alkoxy and —Y-L-X—P(O)(OR^{X})(OR^{Y});
with the proviso that at least one of P¹ and P² is —Y-L-X—P(O)(OR^{X})(OR^{Y});
R^{B} is selected from H and C₁-C₆alkyl;
R^{X} and R^{Y} are independently selected from H and C₁-C₆alkyl;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂;
each p is independently selected from 1, 2, 3, 4, 5 and 6; and
q is selected from 1, 2, 3 and 4.

In some embodiments of formula (K′): P¹ is selected from C₁-C₆alkyl optionally substituted with COOH and —Y-L-X—P(O)(OR^{X})(OR^{Y}); P² is selected from C₁-C₆alkoxy and —Y-L-X—P(O)(OR^{X})(OR^{Y}); R^{B} is C₁-C₆alkyl; X is a covalent bond; L is selected from C₁-C₆alkylene and —((CH₂)ₚO)_{q}(CH₂)ₚ each optionally substituted with 1 to 4 substituents independently selected from halo, OH, C₁-C₄alkyl, —OP(O)(OH)₂ and P(O)(OH)₂; each p is independently selected from 1, 2 and 3; q is selected from 1 and 2.

Formula (F) TLR 7 agonists [14]

The TLR agonist can be a compound according to formula (F):

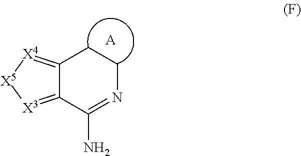

wherein:
X³ is N;
X⁴ is N or CR³
X⁵ is —CR⁴═CR⁵—;
R¹ and R² are H;
R³ is H;
R⁴ and R⁵ are each independently selected from H, halogen, —C(O)OR⁷, —C(O)R⁷, —C(O)N(R¹¹R¹²), —N(R¹¹R¹²), —N(R⁹)₂, —NHN(R⁹)₂, —SR′, —(CH₂)₆OR⁷, —(CH₂)ₙOR⁷, -LR⁸, -LR¹⁰, —OLR⁸, —OLR¹⁰, C₁-C₆alkyl, C₁-C₆heteroalkyl, C₁-C₆halo alkyl, C₂-C₈alkene, C₂-C₅alkyne, C₁-C₆alkoxy, C₁-C₆haloalkoxy, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₈heterocycloalkyl groups of R⁴ and R⁵ are each optionally substituted with 1 to 3 substituents independently selected from halogen, —CN, —NO₂, —R⁷, —OR⁸, —C(O)R⁸, —OC(O)R⁸, —C(O)OR⁸, —N(R⁹)₂, —P(O)(OR⁸)₂, —OP(O)(OR⁸)₂, —P(O)(OR¹⁰)₂. —OP(O)(OR¹⁰)₂, —C(O)N(R⁹)₂, —S(O)₂R⁸, —S(O)R⁸, —S(O)₂N(R⁹)₂, and —NR⁹S(O)₂R⁸;
or, R³ and R⁴, or R⁴ and R⁵, or R⁵ and R⁶, when present on adjacent ring atoms, can optionally be linked together to form a 5-6 membered ring, wherein the 5-6 membered ring is optionally substituted with R⁷;
each L is independently selected from a bond, —(O(CH₂)ₘ)ₜ—, C₁-C₆alkyl, C₂-C₆alkenylene and C₂-C₆alkynylene, wherein the C₁-C₆alkyl, C₂-C₆alkenylene and C₂-C₆alkynylene of L are each optionally substituted with 1 to 4 substituents independently selected from halogen, —R⁸, —OR⁸, —N(R⁹)₂, —P(O)(OR⁸)₂, —OP(O)(OR⁸)₂, —P(O)(OR¹⁰)₂, and —OP(O)(OR¹⁰)₂;
R⁷ is selected from H, C₁-C₆alkyl, aryl, heteroaryl, C₃-C₈cycloalkyl, C₁-C₆heteroalkyl, C₁-C₆haloalkyl, C₂-C₈alkene, C₂-C₈alkyne, C₁-C₆alkoxy, C₁-C₆haloalkoxy, and C₃-C₈heterocycloalkyl, wherein the C₁-C₆alkyl, aryl, heteroaryl, C₃-C₈cycloalkyl, C₁-C₆hetero alkyl, C₁-C₆halo alkyl, C₂-C₈alkene, C₂-C₈alkyne, C₁-C₆alkoxy, C₁-C₆haloalkoxy, and C₃-C₈heterocycloalkyl groups of R⁷ are each optionally substituted with 1 to 3 R¹³ groups, and each R¹³ is independently selected from halogen, —CN, -LR⁹, -LOR⁹, —OLR⁹, —LR¹⁰, -LOR¹⁰, —OLR¹⁰, -LR⁸, -LOR⁸, —OLR⁸, -LSR⁸, -LSR¹⁰, -LC(O)R⁸, -OLC(O)R⁸, -LC(O)OR⁸, -LC(O)R¹⁰, -LOC(O)OR⁸, -LC(O)NR⁹R¹¹, -LC(O)NR⁹R⁸, -LN(R⁹)₂, -LNR⁹R⁸, -LNR⁹R¹⁰, -LC(O)N(R⁹)₂, -LS(O)₂R⁸, -LS(O)R⁸, -LC(O)NR⁸OH, -LNR⁹C(O)R⁸, -LNR⁹C(O)OR⁸, -LS(O)₂N(R⁹)₂, -OLS(O)₂N(R⁹)₂, -LNR⁹S(O)₂R⁸, -LC(O)NR⁹LN(R⁹)₂, -LP(O)(OR⁸)₂, -LOP(O)(OR⁸)₂, —LP(O)(OR¹⁰)₂ and —OLP(O)(OR¹⁰)₂;
each R⁸ is independently selected from H, —CH(R¹⁰)₂, C₁-C₈alkyl, C₂-C₈alkene, C₂-C₈alkyne, C₁-C₆halo alkyl, C₁-C₆alkoxy, C₁-C₆hetero alkyl, C₃-C₈cycloalkyl, C₂-C₈heterocycloalkyl, C₁-C₆hydroxyalkyl and C₁-C₆haloalkoxy, wherein the C₁-C₈alkyl, C₂-C₈alkene, C₂-C₈alkyne, C₁-C₆heteroalkyl, C₁-C₆halo alkyl, C₁-C₆alkoxy, C₃-C₈cycl o alkyl, C₂-C₈heterocycloalkyl, C₁-C₆hydroxyalkyl and C₁-C₆haloalkoxy groups of R⁸ are each optionally substituted with 1 to 3 substituents independently selected from —CN, R¹¹, —OR¹¹, —SR¹¹, —C(O)R¹¹, —OC(O)R¹¹, —C(O)N(R⁹)₂, —C(O)OR¹¹, —NR⁹C(O)R¹¹, —NR⁹R¹°, —NR¹¹R¹², —N(R⁹)₂, —OR⁹, —OR¹⁰, —C(O)NR¹¹R¹², —C(O)NR¹¹OH, —S(O)₂R¹¹, —S(O)R¹¹, —S(O)₂NR¹¹R¹², —NR¹¹S(O)₂R¹¹, —P(O)(OR¹¹)₂, and —OP(O)(OR¹¹)₂;
each R⁹ is independently selected from H, —C(O)R⁸, —C(O)OR⁸, —C(O)R¹⁰, —C(O)OR¹⁰, —S(O)₂R¹⁰, —C₁-C₆ alkyl, C₁-C₆ heteroalkyl and C₃-C₆ cycloalkyl, or each R⁹ is independently a C₁-C₆alkyl that together with N they are attached to form a C₃-C₈heterocycloalkyl, wherein the C₃-C₈heterocycloalkyl ring optionally contains an additional heteroatom selected from N, O and S, and wherein the C₁-C₆ alkyl, C₁-C₆ heteroalkyl, C₃-C₆ cycloalkyl, or C₃-C₈heterocycloalkyl groups of R⁹ are each optionally substituted with 1 to 3 substituents independently selected from —CN, R¹¹, —OR¹¹, —SR¹¹, —C(O)R¹¹, OC(O)R¹¹, —C(O)OR¹¹, —NR¹¹R¹², —C(O)NR¹¹R¹², —C(O)NR¹¹OH, —S(O)₂R¹¹, —S(O)₂NR¹¹R¹², —NR¹¹S(O)₂R¹¹, —P(O)(OR¹¹)₂ and —OP(O)(OR¹¹)₂;
each R¹⁰ is independently selected from aryl, C₃-C₈cycloalkyl, C₃-C₈heterocycloalkyl and heteroaryl, wherein the aryl, C₃-C₈cycloalkyl, C₃-C₈heterocycloalkyl and heteroaryl groups are optionally substituted with 1 to 3 substituents selected from halogen, —R⁸, —OR⁸, —LR⁹, -LOR⁹, —N(R⁹)₂, —NR⁹C(O)R⁸, —NR⁹CO₂R⁸. —CO₂R⁸, —C(O)R⁸ and —C(O)N(R⁹)₂;
R¹¹ and R¹² are independently selected from H, C₁-C₆alkyl, C₁-C₆heteroalkyl, C₁-C₆haloalkyl, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, aryl, heteroaryl, C₃-C₈cycloalkyl, and C₃-C₈heterocycloalkyl groups of R¹¹ and R¹² are each optionally substituted with 1 to 3 substituents independently selected from halogen, —CN, R⁸, —OR⁸, C(O)R⁸, OC(O)R⁸, —C(O)OR⁸, —N(R⁹)₂, —NR⁸C(O)R⁸, —NR⁸C(O)OR⁸, —C(O)N(R⁹)₂, C₃-C₈heterocycloalkyl, —S(O)₂R⁸, —S(O)₂N(R⁹)₂, —NR⁹S(O)₂R⁸, C₁-C₆haloalkyl and C₁-C₆haloalkoxy;
or R¹¹ and R¹² are each independently C₁-C₆alkyl and taken together with the N atom to which they are attached form an optionally substituted C₃-C₈heterocycloalkyl ring optionally containing an additional heteroatom selected from N, O and S;
ring A is an aryl or a heteroaryl, wherein the aryl and heteroaryl groups of Ring A are optionally substituted with 1 to 3 R^{A} groups, wherein each R^{A} is independently selected from —R⁸, —R⁷, —OR⁷, —OR⁸, —R¹⁰, —OR¹⁰, —SR⁸, —NO₂, —CN, —N(R⁹)₂, —NR⁹C(O)R⁸, —NR⁹C(S)R⁸, —NR⁹C(O)N(R⁹)₂, —NR⁹C(S)N(R⁹)₂, —NR⁹CO₂R⁸, —NR⁹NR⁹C(O)R⁸, —NR⁹NR⁹C(O)N(R⁹)₂, —NR⁹NR⁹CO₂R⁸, —C(O)C(O)R⁸, —C(O)CH₂C(O)R⁸, —CO₂R⁸, —(CH₂)CO₂R⁸, —C(O)R⁸, —C(S)R⁸, —C(O)N(R⁹)₂, —C(S)N(R⁹)₂, —OC(O)N(R⁹)₂, —OC(O)R⁸, —C(O)N(OR⁸)R⁸, —C(NOR⁸)R⁸, —S(O)₂R⁸, —S(O)₃R⁸, —SO₂N(R⁹)₂, —S(O)R⁸, —NR⁹SO₂N(R⁹)₂, —NR⁹SO₂R⁸, —P(O)(OR⁸)₂, —OP(O)(OR⁸)₂, —P(O)(OR¹⁰)₂, —OP(O)(OR¹⁰)₂, —N(OR⁸)R⁸, —CH═CHCO₂R⁸, —C(═NH)—N(R⁹)₂, and —(CH₂)NHC(O)R⁸ or two adjacent R^{A} substituents on Ring A form a 5-6 membered ring that contains up to two heteroatoms as ring members;
n is, independently at each occurrence, 0, 1, 2, 3, 4, 5, 6, 7 or 8;
each m is independently selected from 1, 2, 3, 4, 5 and 6, and
t is 1, 2, 3, 4, 5, 6, 7 or 8.

Formulae (C), (D), (E), (G) and (H)

As discussed above, the TLR agonist can be of formula (C), (D), (E), (G) or (H).

The ‘parent’ compounds of formulae (C), (D), (E) and (H) are useful TLR7 agonists (see references 12-15 and 31-47) but are preferably modified herein by attachment of a phosphorus-containing moiety.

In some embodiments of formulae (C), (D) and (E) the compounds have structures according to formulae (C′), (D′) and (E′), shown below:

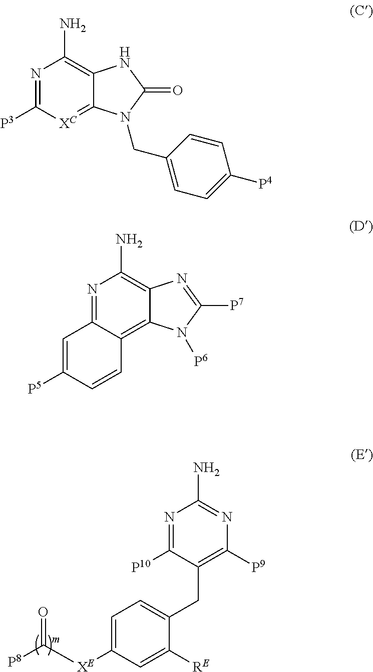

The embodiments of the invention of formulae (C), (D), (E) and (H) also apply to formulae (C′), (D′), (E′) and (H′).

In some embodiments of formulae (C), (D), (E), and (H): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; and q is selected from 1 and 2.

In other embodiments of formula (C): P³ is selected from C₁-C₆alkyl, CF₃, and —((CH₂)ₚO)_{q}(CH₂)ₚOₛ— and —Y-L-X—P(O)(OR^{X})OR^{Y}); P⁴ is selected from —C₁-C₆alkylaryl and —Y-L-X—P(O)(OR^{X})(OR^{Y}); X^{C} is CH; X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; q is 1 or 2.

In other embodiments of formulae (C), (D), (E), and (H): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; and q is selected from 1 and 2.

In other embodiments of formula (C): P³ is selected from C₁-C₆alkyl, CF₃, and —((CH₂)ₚO)_{q}(CH₂)ₚOₛ— and —Y-L-X—P(O)(OR^{X})OR^{Y}); P⁴ is selected from —C₁-C₆alkylaryl and —Y-L-X—P(O)(OR^{X})(OR^{Y}); X^{C} is N; X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; q is selected from 1 and 2.

In other embodiments of formula (D): P⁵ is selected from C₁-C₆alkyl, and —Y-L-X—P(O)(OR^{X})(OR^{Y}).

In other embodiments of formula (D): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; and q is selected from 1 and 2.

In other embodiments of formula (D): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In other embodiments of formula (E): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; and q is selected from 1 and 2.

In other embodiments of formula (E): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; and q is selected from 1 and 2.

In other embodiments of formula (E): X^{E} is CH₂, P⁸ is C₁-C₆alkoxy optionally substituted with —Y-L-X—P(O)(OR^{X})(OR^{Y}).

In other embodiments of formula (E): P⁹ is —NHC₁-C₆alkyl optionally substituted with OH and C₁-C₆alkyl, and —Y-L-X—P(O)(OR^{X})(OR^{Y}).

In some embodiments, a compound of formula (C) is not a compound in which P⁴ is —Y-L-X—P(O)(OR^{X})(OR^{Y}).

In some embodiments, in a compound of formula (C), P⁴ is selected from H, —C₁-C₆alkylaryl.

In some embodiments of formula (H): X^{H1}-X^{H2} is CR^{H2}R^{H3}, R^{H2} and R^{H3} are H, X^{H3} is N, X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; and q is selected from 1 and 2.

In some embodiments of formula (H): X^{H1}-X^{H2} is CR^{H2}R^{H3}, R^{H2} and R^{H3} are H, X^{H3} is N, X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; and q is selected from 1 and 2.

The ‘parent’ compounds of formula (G) are useful TLR8 agonists (see references 16 & 17) but are preferably modified herein by attachment of a phosphorus-containing moiety to permit adsorption. In some embodiments of formula (G), the compounds have structures according to formula (G′);

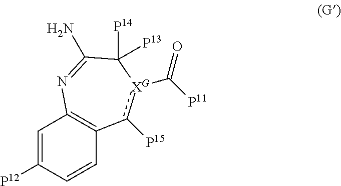

In some embodiments of formula (G) or (G′): X^{G} is C and [symbol] represents a double bond.

In some embodiments of formula (G) or (G′): X is a covalent bond; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂; each p is independently selected from 1, 2 and 3; and q is selected from 1 and 2.

In some embodiments of formula (G) or (G′): X is O; L is selected from C₁-C₆alkylene and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂; each p is independently selected from 1, 2 and 3; and q is selected from 1 and 2.

Pharmaceutical Compositions and Products

The invention provides various immunogenic compositions. These are ideally pharmaceutical compositions suitable for use in humans. Pharmaceutical compositions usually include components in addition to the TLR agonist, insoluble metal salt and/or immunogen e.g. they typically include one or more pharmaceutical carrier(s) and/or excipient(s). A thorough discussion of such components is available in reference 48.

Pharmaceutical compositions are preferably in aqueous form, particularly at the point of administration, but they can also be presented in non-aqueous liquid forms or in dried forms e.g. as gelatin capsules, or as lyophilisates, etc.

Pharmaceutical compositions may include one or more preservatives, such as thiomersal or 2-phenoxyethanol. Mercury-free compositions are preferred, and preservative-free vaccines can be prepared.

Pharmaceutical compositions can include a physiological salt, such as a sodium salt e.g. to control tonicity. Sodium chloride (NaCl) is typical, which may be present at between 1 and 20 mg/ml e.g. 10±2 mg/ml or 9 mg/ml. Other salts that may be present include potassium chloride, potassium dihydrogen phosphate, disodium phosphate dehydrate, magnesium chloride, calcium chloride, etc.

Pharmaceutical compositions can have an osmolality of between 200 mOsm/kg and 400 mOsm/kg, e.g. between 240-360 mOsm/kg, or between 290-310 mOsm/kg. Compositions may be isotonic with humans.

Pharmaceutical compositions may include compounds (with or without an insoluble metal salt) in plain water (e.g. w.f.i.) but will usually include one or more buffers. Typical buffers include: a phosphate buffer (except in the fifteenth aspect); a Tris buffer; a borate buffer; a succinate buffer; a histidine buffer (particularly with an aluminium hydroxide adjuvant); or a citrate buffer. Buffer salt s will typically be included in the 5-20 mM range. If a phosphate buffer is used then the concentration of phosphate ions should, in some embodiments, be <50 mM (see above) e.g. <10 mM.

Pharmaceutical compositions typically have a pH between 5.0 and 9.5 e.g. between 6.0 and 8.0.

Pharmaceutical compositions are preferably sterile.

Pharmaceutical compositions preferably non-pyrogenic e.g. containing <1 EU (endotoxin unit, a standard measure) per dose, and preferably <0.1 EU per dose.

Pharmaceutical compositions are preferably gluten free.

Pharmaceutical compositions can include polygeline (as in the RABIPUR product), but in some embodiments the compositions are polygeline-free.

Pharmaceutical compositions can include human albumin (as in the RABIPUR product), but in preferred embodiments the compositions are free from human albumin, and ideally are substantially free from any serum components.

Pharmaceutical compositions can include antibiotics (typically residual antibiotics from cell culture), such as those seen in the RABIPUR product (neomycin, chlortetracycline, and amphotericin B), but in some embodiments the compositions are antibiotic-free.

Pharmaceutical compositions are suitable for administration to animal (and, in particular, human) patients, and thus include both human and veterinary uses. They may be used in a method of raising an immune response in a patient, comprising the step of administering the composition to the patient.

Compositions may be administered before a subject is exposed to a pathogen and/or after a subject is exposed to a pathogen.

Pharmaceutical compositions may be prepared in unit dose form. In some embodiments a unit dose may have a volume of between 0.05-1.5 ml e.g. about 0.5 ml or about 1.0 ml for intramuscular injection, or smaller volumes (e.g. 0.1 ml) for intradermal injection.

The invention also provides a delivery device (e.g. syringe, nebuliser, sprayer, inhaler, dermal patch, etc.) containing a pharmaceutical composition of the invention e.g. containing a unit dose. This device can be used to administer the composition to a vertebrate subject.

The invention also provides a sterile container (e.g. a vial) containing a pharmaceutical composition of the invention e.g. containing a unit dose.

The invention also provides a unit dose of a pharmaceutical composition of the invention.

The invention also provides a hermetically sealed container containing a pharmaceutical composition of the invention. Suitable containers include e.g. a vial.

The invention also provides a kit comprising first and second kit components, wherein: (i) the first kit component comprises an insoluble metal salt and at least one rabies virus immunogen; and (ii) the second kit component comprises a TLR agonist. The second component ideally does not include an insoluble metal salt and/or does not include a rabies virus immunogen. The first and second components can be combined to provide a composition suitable for administration to a subject.

The invention also provides a kit comprising first and second kit components, wherein: (i) the first kit component comprises an insoluble metal salt and a TLR agonist; and (ii) the second kit component comprises at least one rabies virus immunogen. The second component ideally does not include an insoluble metal salt and/or a TLR agonist. In some embodiments, the second component is lyophilised. The first and second components can be combined to provide a pharmaceutical composition suitable for administration to a subject.

The invention also provides a kit comprising first and second kit components, wherein: (i) the first kit component comprises at least one rabies virus immunogen and a TLR agonist; and (ii) the second kit component comprises an insoluble metal salt. The second component ideally does not include a rabies virus immunogen and/or a TLR agonist. The first and second components can be combined to provide a pharmaceutical composition suitable for administration to a subject.

In some embodiments these kits comprise two vials. In other embodiments they comprise one ready-filled syringe and one vial, with the contents of the syringe being mixed with the contents of the vial prior to injection. A syringe/vial arrangement is useful where the vial's contents are lyophilised. Usually, though, the first and second kit components will both be in aqueous liquid form.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition), although aqueous compositions are preferred. The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. by an inhaler, using a fine powder or a spray. The composition may be prepared as a suppository or pessary. The composition may be prepared for nasal, aural or ocular administration e.g. as a spray or drops. The composition may be in kit form, designed such that a combined composition is reconstituted just prior to administration to a patient. Injectables for intramuscular or intradermal administration are typical.

Compositions comprise an effective amount of a TLR agonist i.e. an amount which, when administered to an individual, either in a single dose or as part of a series, is effective for enhancing the immune response to a co-administered rabies virus immunogen. This amount can vary depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. The amount will fall in a relatively broad range that can be determined through routine trials. An amount of between 1-1000 μg/dose can be used e.g. from 5-100 μg per dose or from 10-100 μg per dose, and ideally ≦300 μg per dose e.g. about 5 μg, 10 μg, 20 μg, 25 μg, 50 μg or 100 μg per dose. Thus the concentration of a TLR agonist in a composition of the invention may be from 2-2000 μg/ml e.g. from 10-200 μg/ml, or about 10, 20, 40, 50, 100 or 200 μg/ml, and ideally ≦600 μg/ml.

Methods of Treatment, and Administration of Immunogenic Compositions

The invention is suitable for raising immune responses in humans, but they may also be useful in non-human animals (in particular mammals, such as dogs, cats, ferrets, rabbits, skunks, or foxes) subjects. Compositions prepared according to the invention may be used to treat both children and adults.

The invention provides a method of raising an immune response in a subject, comprising the step of administering to the subject a composition of the invention. The invention also provides a composition of the invention, for use in a method of raising an immune response in a subject. The invention also provides the use of (i) a TLR agonist as defined herein and (ii) an insoluble metal salt and (iii) a rabies virus immunogen, in the manufacture of a medicament (e.g. a vaccine) for raising an immune response in a subject.

These methods and uses may involve co-administration of anti-rabies immunoglobulin (e.g. the IMOGAM product), which may be from a human or non-human source (e.g. equine immunoglobulin), but where non-human material is used a patient should ideally be first checked for sensitivity to it. Immunoglobulin is generally given in a single dose of 20 IU per kg of body weight for human anti-rabies immunoglobulin, or 40 IU per kg of body weight for equine immunoglobulin (or for F(ab′)₂ products). It is administered at the same time as the first administration of vaccine, but in a different part of the body. All of the immunoglobulin, or as much as anatomically possible to avoid possible compartment syndrome, should be administered into or around the wound site or sites. The remaining immunoglobulin, if any, should be injected intramuscularly at a site distant from the site of vaccine administration.

The immune response stimulated by these methods and uses will generally include an antibody response, preferably a protective antibody response. The immune response can also include a cellular response. Methods for assessing antibody and cellular immune responses after immunisation are well known in the art. For rabies vaccines the standard test measures virusneutralising antibody (e.g. see chapters 15-17 of reference 27). Protective levels for rabies virus are generally accepted to be a neutralising antibody (Nab) blood titer of at least 0.5 IU/ml, calibrated by reference to an international reference serum standard. Various assay techniques for measuring Nab titers are available, including the Rapid Fluorescent Foci Inhibition Test (RFFIT; chapter 15 of ref. 27), the mouse neutralisation test (MNT; chapter 16 of ref. 27), and the fluorescent antibody virus neutralization (FAVN) test [49]. Complete inhibition in RFFIT at a 1:5 dilution is used as a criterion for protection in some countries.

Thus the immune response stimulated by the methods and uses of the invention, when administered to a human subject, should be manifested by a serum Nab titer of at least 0.5 IU/ml (measured 14 days after administration of the composition). Preferred compositions of the invention can achieve this protective efficacy when administered to humans. Ideally, the composition is sufficiently immunogenic to ensure that a patient maintains a serum Nab titer of at least 0.5 IU/ml for at least 5 years after immunisation is completed e.g. for 6 years, 7 years, 8 years, 9 years, 10 years, or more.

Administration of immunogenic compositions of the invention will generally be by injection, and this may be by the subcutaneous, intradermal of the intramuscular route. Intramuscular injection is preferred, although good results can also be achieved by intradermal injection.

Immunogenic compositions of the invention will generally be administered to people at risk of being infected. These include, but are not limited to: veterinarians; animal handlers; laboratory workers (in some fields); people whose activities bring them into frequent contact with rabies virus, or potentially rabid bats, raccoons, skunks, cats, or dogs; people travelling to countries with endemic rabies; international travellers who are likely to come in contact with animals in areas where dog rabies is relatively common (such as in developing countries in Africa, Asia, and Latin America). These people can be infants (e.g. aged 0-2 years), children (e.g. aged 2-12 years), adolescents (e.g. aged 13-18 years), adults (e.g. aged 19-55 year), or the elderly (e.g. aged 56 years or more).

The methods and uses of the invention can use the dosing regimens provided above, and described in more detail below. More generally, immunisation can be by a single dose (particularly when giving a booster) or a multiple dose regimen (particularly for pre-exposure and post-exposure immunisations). Multiple doses will typically be administered at different times, at least 1 week apart (e.g. about 2 weeks, about 3 weeks, about 4 weeks, about 6 weeks, about 8 weeks, about 10 weeks, about 12 weeks, etc.) although more-closely spaced injections can also be used, as can multiple injections at different sites at substantially the same time. Known regimens, which can be used with the invention, include: intramuscular or intradermal injections on days 0, 7 and 28 or days 0, 7 and 21; the ‘Essen’ regimen, in which doses are administered by intramuscular injection on days 0, 3, 7, 14 and 28/30; an abbreviated multisite “2-1-1” schedule, in which one dose is given into each arm at day 0, followed by injection into the deltoid muscle on days 7 and 21; a post-exposure regimen, involving 4 doses injected intramuscularly on days 0, 3, 7 and 14; the “2-2-2-0-2” regiment, which is a 2-site regimen with intradermal injection in both the deltoid and the thigh on days 0, 3, 7 and 28; 2 intramuscular or intradermal injections separated by three days; the “8-0-4-0-1-1” regimen with 8 intradermal doses on day 0, 7 intradermal doses on day 7, then single intradermal doses on days 28 & 90; and a single-visit 4-site intradermal regimen with 4 injections distributed across the left and right deltoids and thighs. Some of these regimens are more suitable for pre-exposure use (e.g. days 0/7/28), and others for post-exposure use (e.g. Essen or 2-1-1 or 8-0-4-0-1-1 or 2-2-2-0-2). Co-administration of immunoglobulin (see above) may also be helpful with some of these regimens (particularly post-exposure regimens), and in some cases equine immunoglobulin might be preferred [50].

In advantageous embodiments of the invention, however, protection can be achieved using fewer doses than these known regimens. For instance, an advantageous pre-exposure regimen using compositions of the invention can involve 1 or 2 doses in total, and an advantageous post-exposure regimen using compositions of the invention can involve 1, 2 or 3 doses in total.

A further way of exploiting the advantages of the invention does not reduce the number of doses which are administered in a multidose schedule, but it is administers them over a shorter period of time. For instance, a 3-dose schedule (ideally for pre-exposure immunisation) can involve administration on days 0m 3 and 7 i.e. all 3 doses are administered within a week.

The invention can also advantageously achieve protection in a human more rapidly than when using known vaccines. For instance, protection might be achieved within 3 weeks or less (e.g. within 2 weeks, or even within 1 week) of (i) a single administration of a composition of the invention or (ii) the first administration in a multi-dose regimen.

In a useful booster embodiment, an immunogenic composition is administered to a patient who has not received a rabies vaccine for at least 6 years.

Chemical Groups

Unless specifically defined elsewhere, the chemical groups discussed herein have the following meaning when used in present specification:

The term “alkyl” includes saturated hydrocarbon residues including:
linear groups up to 10 atoms (C₁-C₁₀), or of up to 6 atoms (C₁-C₆), or of up to 4 atoms (C₁-C₄). Examples of such alkyl groups include, but are not limited, to C₁-methyl, C₂-ethyl, C₃-propyl and C₄-n-butyl.
branched groups of between 3 and 10 atoms (C₃-C₁₀), or of up to 7 atoms (C₃-C₇), or of up to 4 atoms (C₃-C₄). Examples of such alkyl groups include, but are not limited to, C₃-iso-propyl, C₄-sec-butyl, C₄-iso-butyl, C₄-tert-butyl and C₅-neo-pentyl.

The term “alkylene” refers to the divalent hydrocarbon radical derived from an alkyl group, and shall be construed in accordance with the definition above.

The term “alkenyl” includes monounsaturated hydrocarbon residues including:
linear groups of between 2 and 6 atoms (C₂-C₆). Examples of such alkenyl groups include, but are not limited to, C₂-vinyl, C₃-1-propenyl, C₃-allyl, C₄-2-butenyl
branched groups of between 3 and 8 atoms (C₃-C₈). Examples of such alkenyl groups include, but are not limited to, C₄-2-methyl-2-propenyl and C₆-2,3-dimethyl-2-butenyl.

The term alkenylene refers to the divalent hydrocarbon radical derived from an alkenyl group, and shall be construed in accordance with the definition above.

The term “alkoxy” includes O-linked hydrocarbon residues including:
linear groups of between 1 and 6 atoms (C₁-C₆), or of between 1 and 4 atoms (C₁-C₄). Examples of such alkoxy groups include, but are not limited to, C₁-methoxy, C₂-ethoxy, C₃-n-propoxy and C₄-n-butoxy.
branched groups of between 3 and 6 atoms (C₃-C₆) or of between 3 and 4 atoms (C₃-C₄). Examples of such alkoxy groups include, but are not limited to, C₃-iso-propoxy, and C₄-sec-butoxy and tert-butoxy.

Halo is selected from Cl, F, Br and I. Halo is preferably F.

The term “aryl” includes a single or fused aromatic ring system containing 6 or 10 carbon atoms; wherein, unless otherwise stated, each occurrence of aryl may be optionally substituted with up to 5 substituents independently selected from (C₁-C₆)alkyl, (C₁-C₆)alkoxy, OH, halo, CN, COOR¹⁴, CF₃ and NR¹⁴R¹⁵; as defined above. Typically, aryl will be optionally substituted with 1, 2 or 3 substituents. Optional substituents are selected from those stated above. Examples of suitable aryl groups include phenyl and naphthyl (each optionally substituted as stated above). Arylene refers the divalent radical derived from an aryl group, and shall be construed in accordance with the definition above.

The term “heteroaryl” includes a 5, 6, 9 or 10 membered mono- or bi-cyclic aromatic ring, containing 1 or 2 N atoms and, optionally, an NR¹⁴ atom, or one NR¹⁴ atom and an S or an O atom, or one S atom, or one O atom; wherein, unless otherwise stated, said heteroaryl may be optionally substituted with 1, 2 or 3 substituents independently selected from (C₁-C₆)alkyl, (C₁-C₆)alkoxy, OH, halo, CN, COOR¹⁴, CF₃ and NR¹⁴R¹⁵; as defined below. Examples of suitable heteroaryl groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl and isoquinolinyl (optionally substituted as stated above). Heteroarylene refers the divalent radical derived from heteroaryl, and shall be construed in accordance with the definition above.

The term “heterocyclyl” is a C-linked or N-linked 3 to 10 membered non-aromatic, mono- or bi-cyclic ring, wherein said heterocycloalkyl ring contains, where possible, 1, 2 or 3 heteroatoms independently selected from N, NR¹⁴, S(O)_{q} and O; and said heterocycloalkyl ring optionally contains, where possible, 1 or 2 double bonds, and is optionally substituted on carbon with 1 or 2 substituents independently selected from (C₁-C₆)alkyl, (C₁-C₆)alkoxy, OH, CN, CF₃, halo, COOR¹⁴, NR¹⁴R¹⁵ and aryl.

In the above definitions R¹⁴ and R¹⁵ are independently selected from H and (C₁-C₆)alkyl.

When a structural formula is defined with a substituent attached to the core of the molecule by an unspecified, or “floating” bond, for example, as for the group P³ in the case of formula (C), this definition encompasses the cases where the unspecified substituent is attached to any of the atoms on the ring in which the floating bond is located, whilst complying with the allowable valence for that atom.

In the case of compounds of the invention which may exist in tautomeric forms (i.e. in keto or enol forms), for example the compounds of formula (C) or (H), reference to a particular compound optionally includes all such tautomeric forms.

General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x+10%.

Unless specifically stated, a process comprising a step of mixing two or more components does not require any specific order of mixing. Thus components can be mixed in any order. Where there are three components then two components can be combined with each other, and then the combination may be combined with the third component, etc.

As animal (and particularly bovine) materials are typically used in the culture of cells, they should be obtained from sources that are free from transmissible spongiform encaphalopathies (TSEs), and in particular free from bovine spongiform encephalopathy (BSE).

Where a compound is administered to the body as part of a composition then that compound may alternatively be replaced by a suitable prodrug.

Phosphorous-containing groups employed with the invention may exist in a number of protonated and deprotonated forms depending on the pH of the surrounding environment, for example the pH of the solvent in which they are dissolved. Therefore, although a particular form may be illustrated it is intended, unless otherwise mentioned, for these illustrations to merely be representative and not limiting to a specific protonated or deprotonated form. For example, in the case of a phosphate group, this has been illustrated as —OP(O)(OH)₂ but the definition includes the protonated forms —[OP(O)(OH₂)(OH)]⁺ and —[OP(O)(OH₂)₂]²⁺ that may exist in acidic conditions and the deprotonated forms —[OP(O)(OH)(O)]⁻ and [OP(O)(O)₂]²⁻ that may exist in basic conditions.

Compounds disclosed herein can exist as pharmaceutically acceptable salts. Thus, the compounds may be used in the form of their pharmaceutically acceptable salts i.e. physiologically or toxicologically tolerable salt (which includes, when appropriate, pharmaceutically acceptable base addition salts and pharmaceutically acceptable acid addition salts).

### BRIEF DESCRIPTION OF THE DRAWINGS

FIG. 1 shows RFFIT neutralisation titers in mice at days 20 and 35, in mice receiving either 0.1 IU or 1.0 IU of rabies immunogen. The four bars in each group are, from left to right: unadjuvanted; adjuvanted with Al—H; adjuvanted with Al—H and 100 μg K2; adjuvanted with Al—H and 25 μg K2.

FIG. 2 shows RFFIT neutralisation titers in mice at days 20, 35, 49 and 90 in mice receiving 1.0 IU of unadjuvanted rabies immunogen or 0.1 IU of vaccine adjuvanted with Al—H or Al—H/K2. The three four bars in each group are, from left to right: unadjuvanted; adjuvanted with Al—H; adjuvanted with Al—H and 25 μg K2. FIG. 3 shows anti-glycoprotein IgG titers for the same times and groups.

FIG. 4 shows RFFIT neutralisation titers in mice. The left-hand group of 4 bars shows results in mice who received 1.0 IU of immunogen; the right-hand group received 0.1 IU immunogen. In each group the titers are, from left to right: unadjuvanted after 3 doses; Al—H after 2 doses; and Al—H/K2 (25 μg) after 2 doses. FIG. 5 shows anti-glycoprotein IgG titers for the same groups.

FIGS. 6 and 7 show RFFIT Nab titers at days 20, 35, 49, 90 and 180. FIG. 6 shows data with 1.0 IU of immunogen, whereas FIG. 7 shows data for 0.1 IU immunogen. The data are for unadjuvanted (♦), Al—H () or Al—H/K2(25 μg) (▪).

FIG. 8 shows RFFIT Nab titers at day 35 in groups A to H. Open bars show data from mice who received two doses (4wp2); shaded bars show data for mice who received three doses (2wp3).

FIG. 9 shows RFFIT Nab titers after 3 doses of unadjuvanted vaccine (B) or 2 doses of adjuvanted vaccine (C and E). Group C had Al—H alone, whereas group E had Al—H/K2.

FIG. 10 shows anti-rabies neutralisation titers after (A) 2 doses or (B) 3 doses of vaccine. The data are for unadjuvanted (♦), Al—H (▪) or Al—H/K2 (25 μg) (▴).

### MODES FOR CARRYING OUT THE INVENTION

Vaccine Preparation

References 30 and 51 disclose TLR7 agonists having formula (K) as discussed above. One of these compounds, 3-(5-amino-2-(2-methyl-4-(2-(2-(2-phosphonoethoxy)ethoxy)ethoxy)phenethyl)benzo[f]-[1,7]naphthyridin-8-yl)propanoic acid is referred to hereafter as compound “K2”:

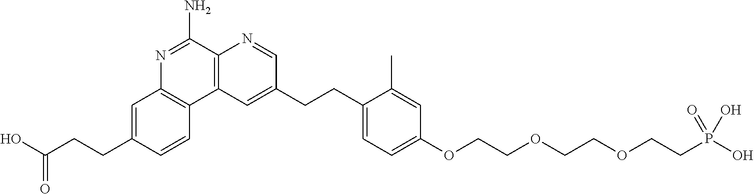 [SMILES: Cc1cc(OCCOCCOCCP(=O)(O)O)ccc1CCc1cnc2c(N)nc3cc(CCC(=O)O)ccc3c2c1]

Compound K2 is added to water at 4 mg/ml, then 1M NaOH is added to ensure full solubilisation, with stirring for 15 minutes at room temperature. This material is added to a suspension of aluminium hydroxide adjuvant (Al—H) to give the desired final concentration. This mixture is shaken for 2 hours at ambient temperature to ensure full adsorption, and then histidine buffer components are added (10 mM histidine buffer, pH 6.5).

The compound can also be used as an arginine salt monohydrate (obtained by mixing 98 mg of the compound with 1.7 ml of 0.1 M arginine in 80/20 methanol/water to give a 57 mg/ml solution, followed by addition of 7 ml ethanol to precipitate the salt) in which case it is seen that the NaOH is not required for solubilisation prior to mixing with the Al—H.

Four different mixtures are prepared, giving a final K2 concentration of 10, 50, 250 or 500 μg/ml (to provide a 1, 5, 25 or 50 μg dose of K2 in a 100 μl dosage volume); the Al—H concentration is always 3 mg/ml. At all strengths >95% of compound K2 is adsorbed to the Al—H. The adsorbed adjuvant is referred to hereafter as “Al—H/K2”.

Rabies virus was grown in purified chicken embryo cell (PCEC) culture, and purified virus was inactivated with β-propiolactone (see, for instance, chapter 28 of reference 27), then purified to produce a vaccine “RV”. The final concentration of virus in RV was ≧2.5 IU/ml.

The RV vaccine was mixed with Al—H or Al—H/K2 (with either 25 μg or 100 μg K2 per dose) to give adjuvanted vaccine.

Immunisation Study A

Balb/c mice received adjuvanted or unadjuvanted RV at a dosage strength of 0.1 IU or 1.0 IU. Vaccines were administered to the mice on days 0, 7 and 21, and immune responses were evaluated on days 20 (2wp2), 35 (2wp3), 49 (4wp3), 90 and 180. Immune responses were assessed by Nab titer (RFFIT) or by anti-glycoprotein IgG titers.

8 groups of mice were as follows, with groups 1 & 2 representing RABIPUR:

| Group | RV (IU) | AI-H (3 mg/mL) | K2 (μg) |
|---|---|---|---|
| 1 | 1.0 | − | − |
| 2 | 0.1 | − | − |
| 3 | 1.0 | + | − |
| 4 | 0.1 | + | − |
| 5 | 1.0 | + | 100 |
| 6 | 0.1 | + | 100 |
| 7 | 1.0 | + | 25 |
| 8 | 0.1 | + | 25 |

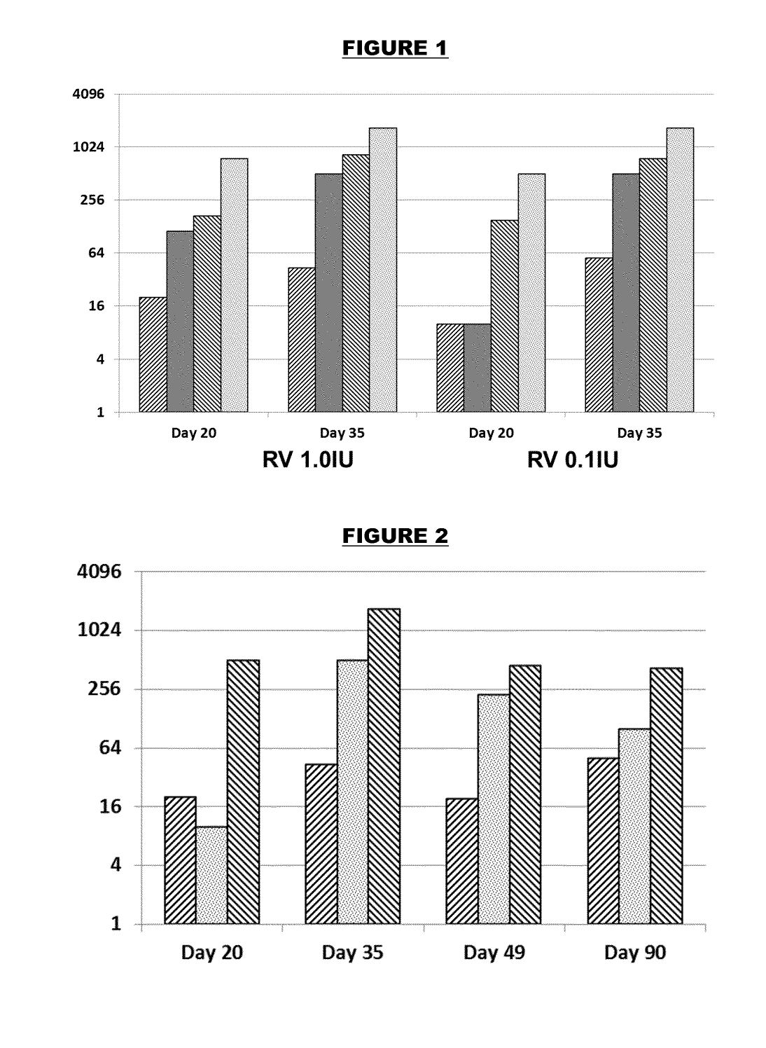

The results in FIG. 1 show that Al—H/K2 enhances Nab titers at 2wp2 and 2wp3 compared to Rabipur (unadjuvanted).

The results in FIGS. 2 and 3 show that Al—H/K2 can be used to achieve good immune responses with lower doses of vaccine, with higher titers achieved using 0.1 IU adjuvanted immunogen than when using unadjuvanted immunogen with a 10× higher dose (1 IU).

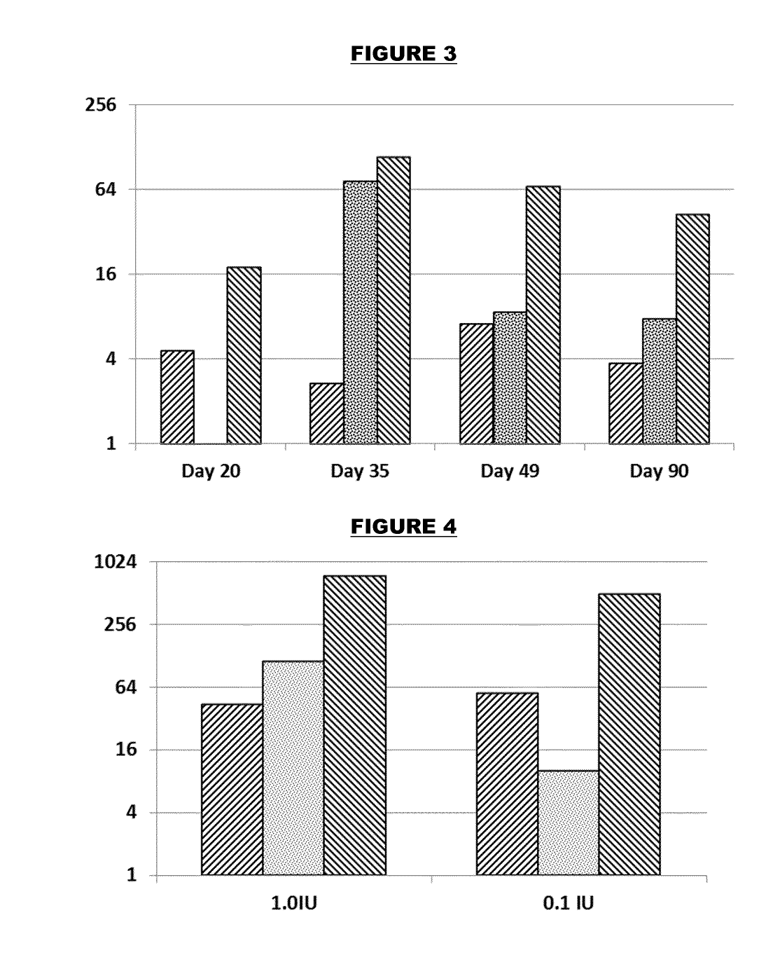

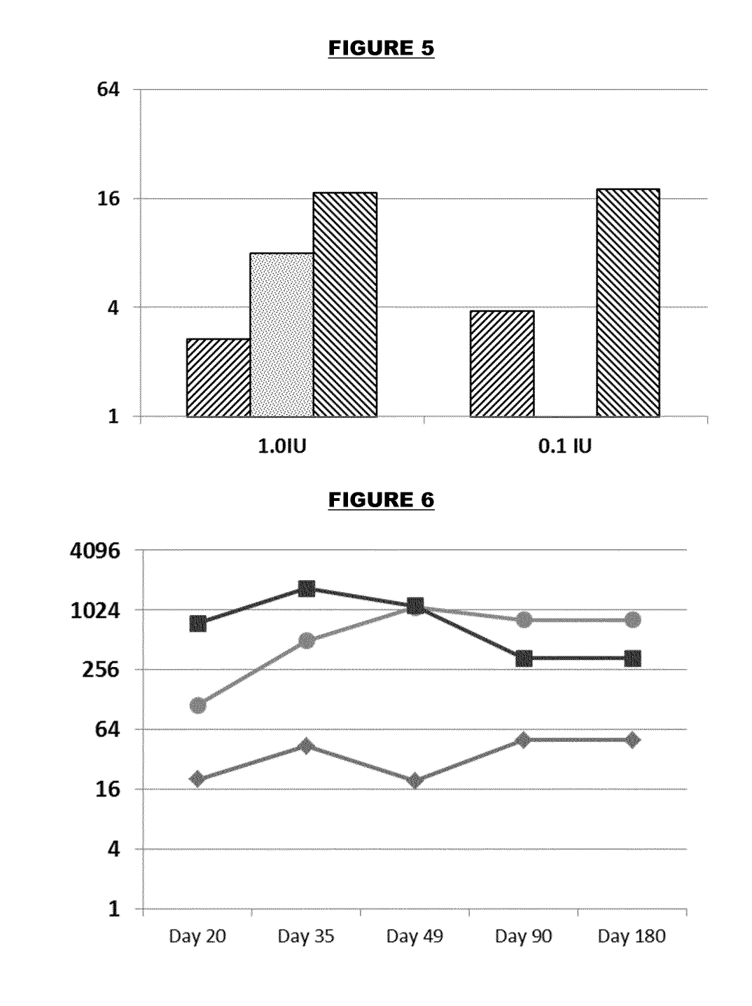

The results in FIGS. 4 and 5 show that Al—H/K2 can be achieve good immune responses using fewer immunisations, with higher titers achieved 2wp2 when using the adjuvanted vaccine than seen at 2wp3 (i.e. after one further dose) when using the unadjuvanted vaccine.

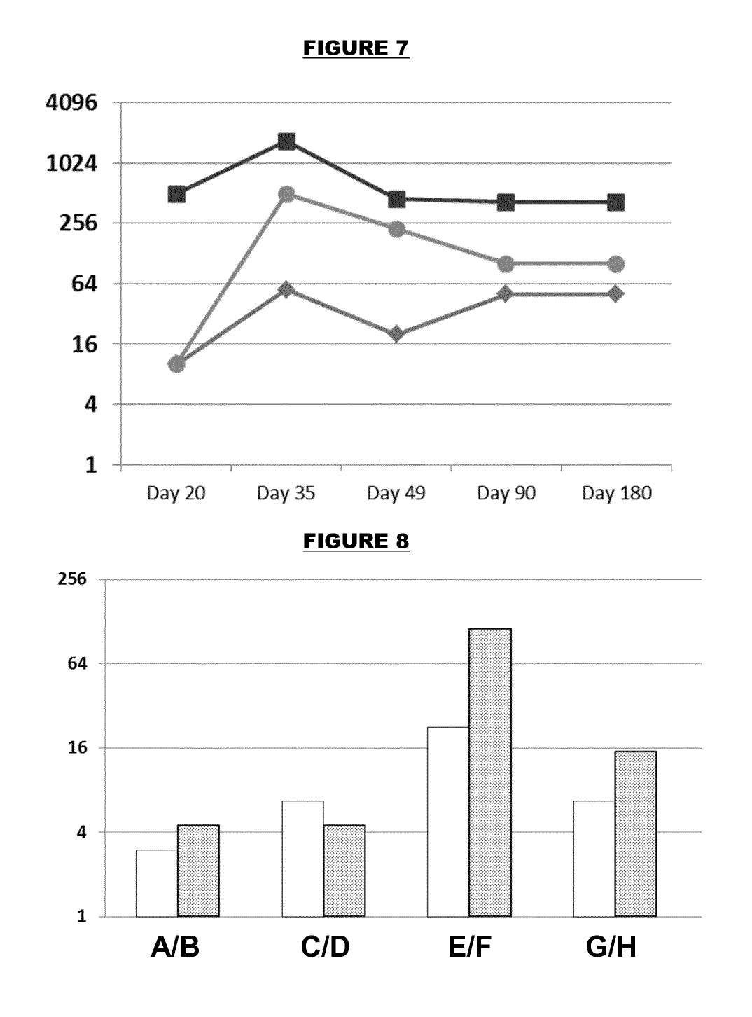

The results in FIGS. 6 and 7 show that Nab titers remain higher in the adjuvanted vaccines at both doses of immunogen, even at days 90 & 180 i.e. months after immunisation was completed.

In conclusion, the use of an aluminium salt and TLR7 agonist: enhances anti-rabies neutralization titers; gives higher anti-rabies titers even with a 10× lower dose of immunogen, thereby permitting more doses to be produced from a batch of purified virus; gives good titers after fewer doses, meaning that fewer, accelerated immunizations are possible; and gives titers with better persistence, meaning that booster immunisations can be less frequent.

Immunisation Study B

Balb/c mice received adjuvanted or unadjuvanted RV at a dosage strength of 0.1 IU. Vaccines were administered to the mice either (i) on days 0 & 7 or (ii) on days 0, 7 & 21. Immune responses were evaluated on days 20 (2wp2), 35 (2wp3), 49 (4wp3), 90 and 180 by Nab titer (RFFIT).

6 groups of mice were as follows, with groups 1 & 2 representing RABIPUR:

| Group | RV (IU) | AI-H (3 mg/mL) | K2 (μg) | Regimen (days) |
|---|---|---|---|---|
| A | 0.1 | − | − | 0, 7 |
| B | 0.1 | − | − | 0, 7, 21 |
| C | 0.1 | + | − | 0, 7 |
| D | 0.1 | + | − | 0, 7, 21 |
| E | 0.1 | + | 25 | 0, 7 |
| F | 0.1 | + | 25 | 0, 7, 21 |

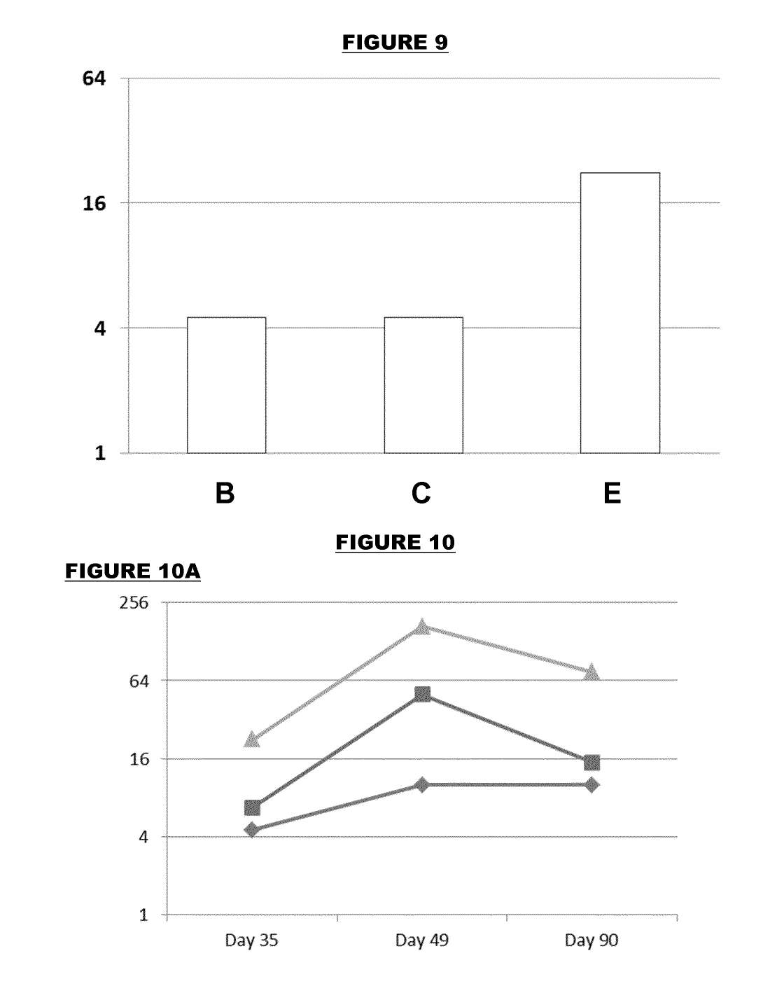

The results in FIG. 8 show that Nab titers are greatly enhanced by Al—H/K2 when compared to Al—H alone. FIG. 9 (RFFIT titers for bleeds taken on day 35) shows that Al—H/K2 can be used to reduce the immunising dose of rabies immunogen, with fewer injections: titers in group C (Al—H, 2 doses) are similar to those in group B (3 unadjuvanted doses), but are much lower than those in group E (2 doses, Al—H/K2 adjuvant).

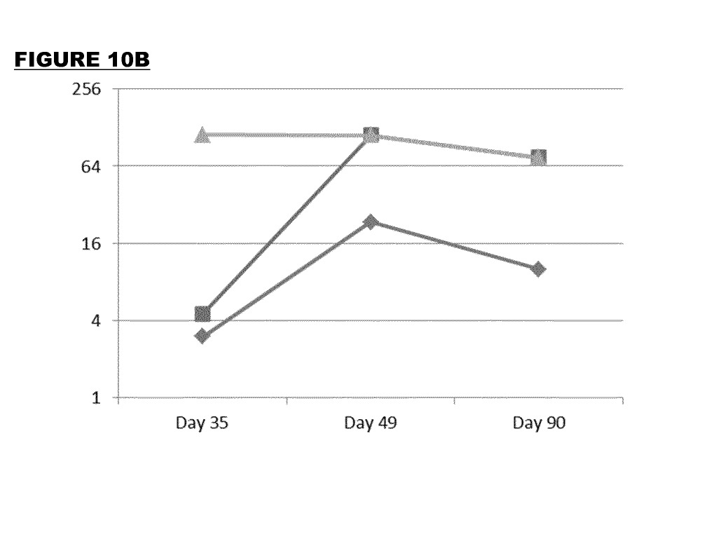

FIG. 10 show Nab titers up till day 90. The titers with Al—H/K2 (▴) are higher than in the other groups, and are also more persistent, both with 2 doses (FIG. 10A) or 3 doses (FIG. 10B).

It will be understood that the invention has been described by way of example only and modifications may be made whilst remaining within the scope and spirit of the invention.

### REFERENCES

[1] Vodopija et al. (1999) Vaccine 17:1739-41.
[2] Toovey (2007) Travel Med Infect Dis 5:327-48.
[3] Côrtes et al. (1993) Rev. sci. tech. Off. Mt. Epiz. 12:941-55.
[4] Berlin et al. (1990) Am J Public Health 80:476-8.
[5] Lin & Perrin (1999) Zhonghua Shi Yan He Lin Chuang Bing Du Xue Za Zhi. 13:133-5.
[6] WO2012/031140.
[7] Burrell et al. (1999) Vaccine 17:2599-603.
[8] Vaccine Design . . . (1995) eds. Powell & Newman. ISBN: 030644867X. Plenum.
[9] Clausi et al. (2008) J Pharm Sci DOI 10.1002/jps.21390.
[10] Rosenberg et al. (2010) J Immunol 184:136.20.
[11] U.S. Pat. No. 4,666,886.
[12] WO2009/118296.
[13] WO2008/005555.
[14] WO2009/111337.
[15] WO2009/067081.
[16] WO2007/040840.
[17] WO2010/014913.
[18] GB-A-2220211.
[19] Myers et al. (1990) pages 145-156 of Cellular and molecular aspects of endotoxin reactions.
[20] Ulrich (2000) Chapter 16 (pages 273-282) of Vaccine Adjuvants: Preparation Methods and Research Protocols (ed. O'Hagan, ISBN 978-0-89603-735-9).
[21] Johnson et al. (1999) J Med Chem 42:4640-9.
[22] Baldrick et al. (2002) Regulatory Toxicol Pharmacol 35:398-413.
[23] WO 94/21292.
[24] Vaccines (Plotkin, Orenstein, Offit) Fifth edition, 2008. ISBN 978-1-4160-3611-1.
[25] WHO Technical Report Series, No. 941 (2007), Annex 2.
[26] WHO Technical Report Series, No. 824 (1992), Annex 1.
[27] Laboratory techniques in rabies (1996) eds. Meslin et al. ISBN 92 4 154479 1.
[28] EP-A-1593392.
[29] WO2011/119759.
[30] WO2011/027222.
[31] WO2007/034917.
[32] WO2007/034173.
[33] WO2008/114817.
[34] US2009-0105212.
[35] US2009-0118263.
[36] US2009-0143400.
[37] US2009-0192153.
[38] WO2007/093901.
[39] WO2009/019553.
[40] US2009/0221631.
[41] WO2008/004948.
[42] WO2008/135791.
[43] US2009/0099216.
[44] US2009/0202484.
[45] WO2008/101867.
[46] WO2010/077613.
[47] US2010/0143301.
[48] Remington: The Science and Practice of Pharmacy (Gennaro, 2000; 20th edition, ISBN: 0683306472)
[49] Cliquet et al. (1998) J Immunol Meth 212:79-87.
[50] Lang et al. (1998) Bulletin WHO 76:491-5.
[51] WO2011/049677.

1. An immunogenic composition comprising (i) an insoluble metal salt (ii) a human TLR agonist and (iii) a rabies virus immunogen.

2. The immunogenic composition of claim 1, wherein the insoluble metal salt is an insoluble aluminium salt and the TLR agonist is a TLR7 agonist.

3. An immunogenic composition comprising (i) an aluminium hydroxide adjuvant (ii) compound ‘K2’ or a pharmaceutically acceptable salt thereof, adsorbed to the aluminium hydroxide and (iii) an inactivated rabies virus, wherein compound K2 is according to the formula:

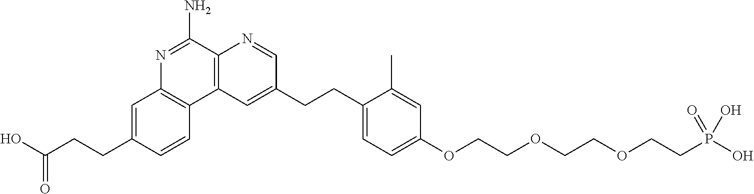 [SMILES: Cc1cc(OCCOCCOCCP(=O)(O)O)ccc1CCc1cnc2c(N)nc3cc(CCC(=O)O)ccc3c2c1]

4. (canceled)

5. (canceled)

6. (canceled)

7. (canceled)

8. (canceled)

9. (canceled)

10. The composition of claim 1, wherein the TLR agonist is adsorbed to the insoluble metal salt.

11. The composition of claim 10, wherein the TLR agonist and the rabies immunogen are both adsorbed to the metal salt.

12. The composition of claim 1, wherein the TLR agonist is a compound of formula ‘K’, or a pharmaceutically acceptable salt thereof, where formula K is: 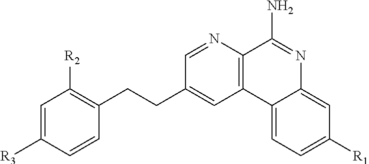
wherein:
R¹ is H, C₁-C₆alkyl, —C(R⁵)₂OH, -L¹R⁵, -L¹R⁶, -L²R⁵, -L²R⁶, —OL²R⁵, or —OL²R⁶;
L¹ is C(O)— or —O—;
L² is C₁-C₆alkylene, C₂-C₆alkenylene, arylene, heteroarylene or —((CR⁴R⁴)ₚO)_{q}(CH₂)ₚ—, wherein the C₁-C₆alkylene and C₇-C₆alkenylene of L² are optionally substituted with 1 to 4 fluoro groups;
each L³ is independently selected from C₁-C₆alkylene and —((CR⁴R⁴)ₚO)_{q}(CH₂)ₚ—, wherein the C₁-C₆alkylene of L³ is optionally substituted with 1 to 4 fluoro groups;
L⁴ is arylene or heteroarylene;
R² is H or C₁-C₆alkyl;
R³ is selected from C₁-C₄alkyl, L³R⁵, -L¹R⁵, -L³R⁷, -L³L⁴L³R⁷, -L³L⁴R⁵, -L³L⁴L³R⁵, —OL³R⁵, —OL³R², —OL³L⁴R⁷, —OL³L⁴L³R⁷, —OR⁸, —OL³L⁴R⁵, —OL³L⁴L³R⁵ and —C(R⁵)₂OH;
each R⁴ is independently selected from H and fluoro;
R⁵ is —P(O)(OR⁹)₂,
R⁶ is CF₂P(O)(OR⁹)₂ or —C(O)OR¹⁰;
R⁷ is —CF₂P(O)(OR⁹)₂ or —C(O)OR¹⁰;
R⁸ is H or C₁-C₄alkyl;
each R⁹ is independently selected from H and C₁-C₆alkyl;
R¹⁰ is H or C₁-C₄alkyl;
each p is independently selected from 1, 2, 3, 4, 5 and 6, and
q is 1, 2, 3 or 4.

13. The composition of claim 12, wherein the TLR agonist is compound K2, wherein compound K2 is according to formula:

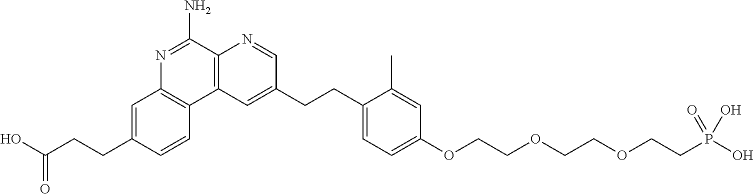 [SMILES: Cc1cc(OCCOCCOCCP(=O)(O)O)ccc1CCc1cnc2c(N)nc3cc(CCC(=O)O)ccc3c2c1]

14. The composition of claim 12, wherein the composition is (a) not lyophilized, or (b) is in aqueous form but is not prepared by aqueous reconstitution of a lyophilisate.

15. The composition of claim 12, wherein the composition (a) does not include a disaccharide or (b) has <20 mg/ml of disaccharide.

16. The composition of claim 12, wherein the composition is free from polygeline.

17. The composition of claim 12, wherein the composition does not include human albumin.

18. The composition of claim 12, wherein the composition is antibiotic-free.

19. (canceled)

20. (canceled)

21. The composition of claim 1, wherein the TLR agonist is selected from a compound according to formula (C), (D), (E), (F), (G), (H), (I), (II), (J) or (K), where formula (C), (D), (E) and (H) are: 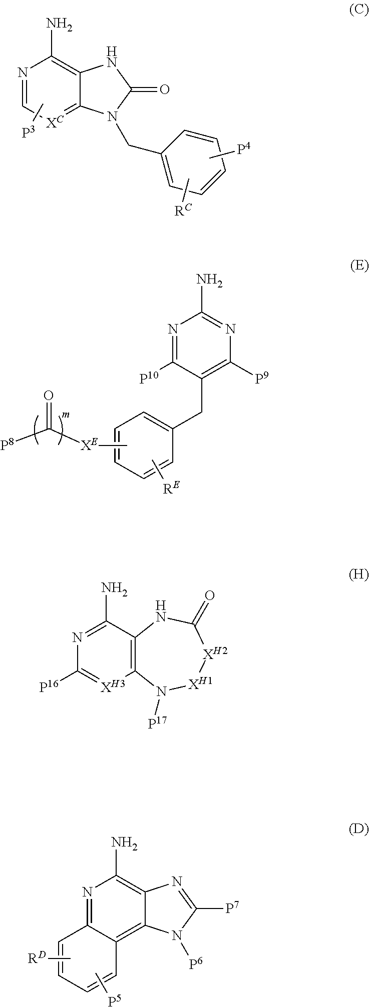
wherein:
(a) P³ is selected from H, C₁-C₆alkyl, CF₃, and —((CH₂)ₚO)_{q}(CH₂)ₚOₛ— and —Y-L-X—P(O)(OR^{X})(OR^{Y}); and P⁴ is selected from H, C₁-C₆alkyl, —C₁-C₆alkylaryl and —Y-L-X—P(O)(OR^{X})(OR^{Y}); with the proviso that at least one of P³ and P⁴ is —Y-L-X—P(O)(OR^{X})(OR^{Y}),
(b) P⁵ is selected from H, C₁-C₆alkyl, and —Y-L-X—P(O)(OR^{X})(OR^{Y}); P⁶ is selected from H, C₁-C₆alkyl each optionally substituted with 1 to 3 substituents selected from C₁-C₄alkyl and OH, and —Y-L-X—P(O)(OR^{X})(OR^{Y}); and P⁷ is selected from H, C₁-C₆alkyl, —((CH₂)ₚO)_{q}(CH₂)ₚOₛ—, —NHC₁-C₆alkyl and —Y-L-X—P(O)(OR^{X})(OR^{Y}); with the proviso that at least one of P⁵, P⁶ and P⁷ is —Y-L-X—P(O)(OR^{X})(OR^{Y});
(c) P⁸ is selected from H, C₁-C₆alkyl, C₁-C₆alkoxy, —NHC₁-C₆alkyl each optionally substituted with OH, and —Y-L-X—P(O)(OR^{X})(OR^{Y}); and P⁹ and P¹⁰ are each independently selected from H, C₁-C₆alkyl, C₁-C₆alkoxy, —NHC₁-C₆alkyl each optionally substituted with OH and C₁-C₆alkyl, and —Y-L-X—P(O)(OR^{X})(OR^{Y}); with the proviso that at least one of P⁸, P⁹ or P¹⁰ is —Y-L-X—P(O)(OR^{X})(OR^{Y});
(d) P¹⁶ and each P¹⁸ are each independently selected from H, C₁-C₆alkyl, and —Y-L-X—P(O)(OR^{X})(OR^{Y}); P¹⁷ is selected from H, C₁-C₆alkyl, aryl, heteroaryl, C₁-C₆alkylaryl, C₁-C₆alkyl heteroaryl, C₁-C₆alkylaryl-Y-L-X—P(O)(OR^{X})(OR^{Y}) and —Y-L-X—P(O)(OR^{X})(OR^{Y}), each optionally substituted with 1 to 2 substituents selected from C₁-C₆alkyl or heterocyclyl with the proviso that at least one of P¹⁶, P¹⁷ or a P¹⁸ contains a —Y-L-X—P(O)(OR^{X})(OR^{Y}) moiety;
R^{X} and R^{Y} are independently selected from H and C₁-C₆alkyl;
R^{C}, R^{D} and R^{H} are each independently selected from H and C₁-C₆alkyl;
X^{C} is selected from CH and N;
R^{E} is selected from H, C₁-C₆alkyl, C₁-C₆alkoxy, C(O)C₁-C₆alkyl, halogen and —((CH₂)ₚO)_{q}(CH₂)ₚ—;
X^{E} is selected from a covalent bond, CR^{E2}R^{E3} and NR^{E4};
R^{E2}, R^{E3} and R^{E4} are independently selected from H and C₁-C₆alkyl;
X^{H1}-X^{H2} is selected from —CR^{H2}R^{H3}, —CR^{H2}R^{H3}—CR^{H2}R^{H3}, —C(O)CR^{H2}R^{H13}—, —C(O)CR^{H2}R^{H3}, —CR^{H2}R^{H3}C(O)—, —NR^{H4}C(O)—, C(O)NR^{H4}—, CR^{H2}R^{H3}S(O)₂ and —CR^{H4}═CR^{H2};
R^{H2}, R^{H3} and R^{H4} are each independently selected from H C₁-C₆alkyl and P¹⁸;
X^{H3} is selected from N and CN;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and —P(O)(OH)₂;
m is selected from 0 or 1;
each p is independently selected from 1, 2, 3, 4, 5 and 6;
q is selected from 1, 2, 3 and 4; and
s is selected from 0 and 1;
where formula (F) is: 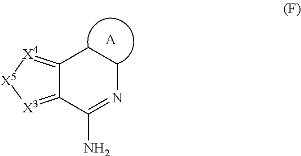
wherein:
X³ is N;
X⁴ is N or CR³
X⁵ is —CR⁴═CR⁵—;
R¹ and R² are H;
R³ is H;
R⁴ and R⁵ are each independently selected from H, halogen, —C(O)OR⁷, —C(O)R⁷, —C(O)N(R¹¹R¹²), —N(R¹¹R¹²), —N(R⁹)₂, —NHN(R⁹)₂, —SR⁷, —(CH₂)ₙOR⁷, —(CH₂)ₙR⁷, -LR⁸, -LR¹⁰, —OLR⁸, —OLR¹⁰, C₁-C₆alkyl, C₁-C₆heteroalkyl, C₁-C₆haloalkyl, C₂-C₈alkene, C₂-C₈alkyne, C₁-C₆alkoxy, C₁-C₆haloalkoxy, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, C₂-C₈alkene C₂-C₆alkyne, C₁-C₆alkoxy, C₁-C₆haloalkoxy, aryl, heteroaryl, C₃-C₈cycloalkyl, and C₃-C₈heterocycloalkyl groups of R⁴ and R⁵ are each optionally substituted with 1 to 3 substituents independently selected from halogen, —CN, —NO₂, —R⁷, —OR⁸, —C(O)R⁸, —OC(O)R⁸, —C(O)OR⁸, —N(R⁹)₂, —P(O)(OR⁸)₂, —OP(O)(OR⁸)₂, —P(O)(OR¹⁰)₂, —OP(O)(OR¹⁰)₂, —C(O)N(R⁹)₂, —S(O)₂R⁸, —S(O)R⁸, —S(O)₂N(R⁹)₂, and —NR⁹S(O)₂R⁸;
or, R³ and R⁴, or R⁴ and R⁵, or R⁵ and R⁶, when present on adjacent ring atoms, can optionally be linked together to form a 5-6 membered ring, wherein the 5-6 membered ring is optionally substituted with R⁷;
each L is independently selected from a bond, —(O(CH₂)ₘ)ₜ—, C₁-C₆alkyl, C₂-C₆alkenylene and C₂-C₆alkynylene, wherein the C₁-C₆alkyl, C₂-C₆alkenylene and C₂-C₆alkynylene of L are each optionally substituted with 1 to 4 substituents independently selected from halogen, —R⁸, —OR⁸, —N(R⁹)₂, —P(O)(OR⁸)₂, —OP(O)(OR⁸)₂, —P(O)(OR¹⁰)₂, and —OP(O)(OR¹⁰)₂;
R⁷ is selected from H, C₁-C₆alkyl, aryl, heteroaryl, C₃-C₈cycloalkyl, C₁-C₆heteroalkyl, C₁-C₆haloalkyl, C₂-C₈alkene, C₂-C₈alkyne, C₁-C₆alkoxy, C₁-C₆haloalkoxy, and C₃-C₈heterocycloalkyl, wherein the C₁-C₆alkyl, aryl, heteroaryl, C₃-C₈cycloalkyl, C₁-C₆heteroalkyl, C₁-C₆haloalkyl, C₂-C₈alkene C₂-C₈alkyne, C₁-C₆alkoxy, C₁-C₆haloalkoxy, and C₃-C₈heterocycloalkyl groups of R⁷ are each optionally substituted with 1 to 3 R¹³ groups, and each R¹³ is independently selected from halogen, —CN, -LR⁹, -LOR⁹, —OLR⁹, -LR¹⁰, -LOR¹⁰, —OLR¹⁰, -LR⁸, -LOR⁸, —OLR⁸, -LSR⁸, -LSR¹⁰, -LC(O)R⁸, -OLC(O)R⁸, -LC(O)OR⁸, -LC(O)R¹⁰, -LOC(O)OR⁸, -LC(O)NR⁹R¹¹, -LC(O)NR⁹R⁸, -LN(R⁹)₂, -LNR⁹R⁸, -LNR⁹R¹⁰, -LC(O)N(R⁹)₂, -LS(O)₂R⁸, -LS(O)R⁸, -LC(O)NR⁸OH, -LNR⁹C(O)R⁸, -LNR⁹C(O)OR⁸, -LS(O)₂N(R⁹)₂, —OLS(O)₂N(R⁹)₂, -LNR⁹S(O)₂R⁸, -LC(O)NR⁹LN(R⁹)₂, -LP(O)(OR⁸)₂, -LOP(O)(OR⁸)₂, -LP(O)(OR¹⁰)₂ and -OLP(O)(OR¹⁰)₂;
each R⁸ is independently selected from H, —CH(R¹⁰)₂, C₁-C₈alkyl, C₂-C₈alkene C₂-C₈alkyne, C₁-C₆haloalkyl, C₁-C₆alkoxy, C₁-C₆heteroalkyl, C₃-C₈cycloalkyl, C₂-C₈heterocycloalkyl, C₁-C₆hydroxyalkyl and C₁-C₆haloalkoxy, wherein the C₁-C₈alkyl, C₂-C₈alkene, C₂-C₈alkyne, C₁-C₆heteroalkyl, C₁-C₆haloalkyl, C₁-C₆alkoxy, C₃-C₈cycloalkyl, C₂-C₈heterocycloalkyl, C₁-C₆hydroxyalkyl and C₁-C₆haloalkoxy groups of R⁸ are each optionally substituted with 1 to 3 substituents independently selected from —CN, R¹¹, —OR¹¹, —SR¹¹, —C(O)R¹¹, —OC(O)R¹¹, —C(O)N(R⁹)₂, —C(O)OR¹¹, —NR⁹C(O)R¹¹, —NR⁹R¹⁰, —NR¹¹R¹², —N(R⁹)₂, OR⁹, —OR¹⁰, —C(O)NR¹¹R¹², —C(O)NR¹¹OH, —S(O)₂R¹¹, —S(O)R¹¹, —S(O)₂NR¹¹R¹², —NR¹¹S(O)₂R¹¹, —P(O)(OR¹¹)₂, and —OP(O)(OR¹¹)₂;
each R⁹ is independently selected from H, —C(O)R⁸, —C(O)OR⁸, —C(O)R¹⁰, —C(O)OR¹⁰, —S(O)₂R¹⁰—C₁-C₆ alkyl, C₁-C₆ heteroalkyl and C₃-C₆ cycloalkyl, or each R⁹ is independently a C₁-C₆alkyl that together with N they are attached to form a C₃-C₈heterocycloalkyl, wherein the C₃-C₈heterocycloalkyl ring optionally contains an additional heteroatom selected from N, O and S, and wherein the C₁-C₆ alkyl, C₁-C₆ heteroalkyl, C₃-C₆ cycloalkyl, or C₃-C₈heterocycloalkyl groups of R⁹ are each optionally substituted with 1 to 3 substituents independently selected from —CN, R¹¹, —OR¹¹, —SR¹¹, —C(O)R¹¹, OC(O)R¹¹, —C(O)OR¹¹, —NR¹¹R¹², —C(O)NR¹¹R¹², —C(O)NR¹¹OH, —S(O)₂R¹¹, —S(O)R¹¹, —S(O)₂NR¹¹R¹², —NR¹¹S(O)₂R¹¹, —P(O)(OR¹¹)₂ and —OP(O)(OR¹¹)₂;
each R¹⁰ is independently selected from aryl, C₃-C₈cycloalkyl, C₃-C₈heterocycloalkyl and heteroaryl, wherein the aryl, C₃-C₈cycloalkyl, C₃-C₈heterocycloalkyl and heteroaryl groups are optionally substituted with 1 to 3 substituents selected from halogen —R⁸, —OR⁸, -LR⁹, -LOR⁹, —N(R⁹)₂, —NR⁹C(O)R⁸, —NR⁹CO₂R⁸, —CO₂R⁸, —C(O)R⁸ and —C(O)N(R⁹)₂;
R¹¹ and R¹² are independently selected from H, C₁-C₆alkyl, C₁-C₆heteroalkyl, C₁-C₆haloalkyl, aryl, heteroaryl, C₃-C₈cycloalkyl, and C₃-C₈heterocycloalkyl, wherein the C₁-C₆alkyl, C₁-C₆heteroalkyl, C₁-C₆haloalkyl, aryl, heteroaryl, C₃-C₈cycloalkyl, and C₃-C₈heterocycloalkyl groups of R¹¹ and R¹² are each optionally substituted with 1 to 3 substituents independently selected from halogen, —CN, R⁸, —OR⁸, C(O)R⁸, OC(O)R⁸, —C(O)OR⁸, —N(R⁹)₂, —NR⁸C(O)R⁸, —NR⁸C(O)OR⁸, —C(O)N(R⁹)₂, C₃-C₈heterocycloalkyl, —S(O)₂R⁸, —S(O)₂N(R⁹)₂, —NR⁹S(O)₂R⁸, C₁-C₆haloalkyl and C₁-C₆haloalkoxy;
or R¹¹ and R¹² are each independently C₁-C₆alkyl and taken together with the N atom to which they are attached form an optionally substituted C₃-C₈heterocycloalkyl ring optionally containing an additional heteroatom selected from N, O and S;
ring A is an aryl or a heteroaryl, wherein the aryl and heteroaryl groups of Ring A are optionally substituted with 1 to 3 R^{A} groups, wherein each R^{A} is independently selected from —R⁸, —R⁷, —OR⁷, —OR⁸, —R¹⁰, —OR¹⁰, —SR⁸, —NO₂, —CN, —N(R⁹)₂, —NR⁹C(O)R⁸, —NR⁹C(S)R⁸, —NR⁹C(O)N(R⁹)₂,—NR⁹C(S)N(R⁹)₂, —NR⁹CO₂R⁸, —NR⁹NR⁹C(O)R⁸, —NR⁹NR⁹C(O)N(R⁹)₂, —NR⁹NR⁹CO₂R⁸, —C(O)C(O)R⁸, —C(O)CH₂C(O)R⁸, —CO₂R⁸, —(CH₂)ₙCO₂R⁸, —C(O)R⁸, —C(S)R⁸, —C(O)N(R⁹)₂, —C(S)N(R⁹)₂, —OC(O)N(R⁹)₂, —OC(O)R⁸, —C(O)N(OR⁸)R⁸, —C(NOR⁸)R⁸, —S(O)₂R⁸, —S(O)₃R⁸, —SO₂N(R⁹)₂, —S(O)R⁸, —NR⁹SO₂N(R⁹)₂, —NR⁹SO₂R⁸, —P(O)(OR⁸)₂, -0P(O)(OR⁸)₂, —P(O)(OR¹⁰)₂, —OP(O)(OR¹⁰)₂, —N(0R⁸)R⁸, —CH═CHCO₂R⁸, —C(═NH)—N(R⁹)₂, and —(CH₂)ₙNHC(O)R⁸ or two adjacent R^{A} substituents on Ring A form a 5-6 membered ring that contains up to two heteroatoms as ring members;
n is, independently at each occurrence, 0, 1, 2, 3, 4, 5, 6, 7 or 8;
each m is independently selected from 1, 2, 3, 4, 5 and 6, and
t is 1, 2, 3, 4, 5, 6, 7 or 8;
where formula (G) is: 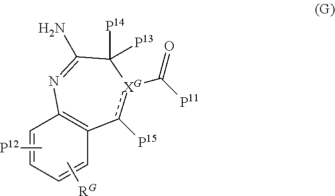
wherein:
P¹¹ is selected from H, C₁-C₆alkyl, C₁-C₆ alkoxy, NR^{V}R^{W} and —Y-L-X—P(O)(OR^{X})(OR^{Y});
P¹² is selected from H, C₁-C₆alkyl, aryl optionally substituted by —C(O)NR^{V}R^{W}, and —Y-L-X—P(O)(OR^{X})(OR^{Y});
P¹³, P¹⁴ and P¹⁵ are independently selected from H, C₁-C₆alkyl, C₁-C₆ alkoxy and —Y-L-X—P(O)(OR^{X})(OR^{Y});
with the proviso that at least one of P¹¹, P¹², P¹³, P¹⁴ or P¹⁵ is —Y-L-X—P(O)(OR^{X})(OR^{Y});
R^{V} and R^{W} are independently selected from H, C₁-C₆alkyl or together with the nitrogen atom to which they are attached form a 4 to 7 remembered heterocyclic ring;
X^{G} is selected from C, CH and N; [symbol]
represents an optional double bond, wherein X^{G} is C if [symbol] is a double bond; and
R^{G} is selected from H and C₁-C₆alkyl;
X is selected from a covalent bond, O and NH;
Y is selected from a covalent bond, O, C(O), S and NH;
L is selected from, a covalent bond C₁-C₆alkylene, C₁-C₆alkenylene, arylene, heteroarylene, C₁-C₆alkyleneoxy and —((CH₂)ₚO)_{q}(CH₂)ₚ— each optionally substituted with 1 to 4 substituents independently selected from halo, OH, C₁-C₄alkyl, —OP(O)(OH)₂ and P(O)(OH)₂;
each p is independently selected from 1, 2, 3, 4, 5 and 6 and
q is selected from 1, 2, 3 and 4;
where formula (I) and (II) are: 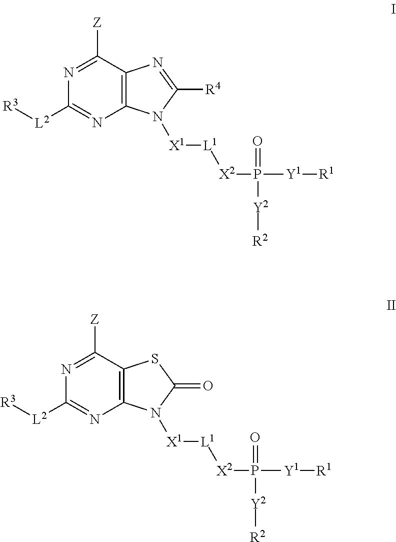
wherein:
Z is —NH₂ or —OH;
X¹ is alkylene, substituted alkylene, alkenylene, substituted alkenylene, alkynylene, substituted alkynylene, carbocyclylene, substituted carbocyclylene, heterocyclylene, or substituted heterocyclylene;
L¹ is a covalent bond, arylene, substituted arylene, heterocyclylene, substituted heterocyclylene, carbocyclylene, substituted carbocyclylene, —S—, —S(O)—, S(O)₂, —NR⁵—, or —O—
X² is a covalent bond, alkylene, or substituted alkylene;
L² is NR⁵—, —N(R⁵)C(O)—, —O—, —S—, —S(O)—, S(O)₂, or a covalent bond;
R³ is H, alkyl, substituted alkyl, heteroalkyl, substituted heteroalkyl, alkenyl, substituted alkenyl, aryl, substituted aryl, arylalkyl, substituted arylalkyl, heterocyclyl, substituted heterocyclyl, heterocyclylalkyl, or substituted heterocyclylalkyl;
Y¹ and Y² are each independently a covalent bond, —O— or —NR⁵—; or —Y¹—R¹ and —Y²—R² are each independently —O—N═C(R⁶R⁷);
R¹ and R² are each independently H, alkyl, substituted alkyl, carbocyclyl, substituted carbocyclyl, heterocyclyl, substituted heterocyclyl, alkenyl, substituted alkenyl, alkynyl, substituted alkynyl, arylalkyl, substituted arylalkyl, heterocyclylalkyl, substituted heterocyclylalkyl, -alkylene-C(O)—O—R⁵, (substituted alkylene)-C(O)—O—R⁵, -alkylene-O—C(O)—R⁵, -(substituted alkylene)-O—C(O)—R⁵, -alkylene-O—C(O)—O—R⁵, or -(substituted alkylene)-O—C(O)—O—R⁵
R⁴ is H, halogen, —OH, —O-alkyl, —O-alkylene-O—C(O)—O—R⁵, —O—C(O)—O—R⁵, —SH, or —NH(R⁵);
each R⁵, R⁶, and R⁷ are independently H, alkyl, substituted alkyl, carbocyclyl, substituted carbocyclyl, heterocyclyl, substituted heterocyclyl, alkenyl, substituted alkenyl, alkynyl, substituted alkynyl, arylalkyl, substituted arylalkyl, heterocyclylalkyl, or substituted heterocyclylalkyl;
where formula (J) is: 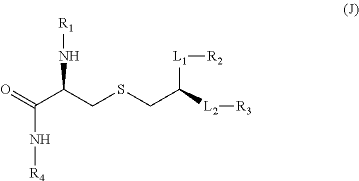
wherein:
R¹ is H, —C(O)—C₇-C₁₈alkyl or —C(O)—C₁-C₆alkyl;
R² is C₂-C₁₈alkyl;
R³ is C₂-C₁₈alkyl;
L₁ is —CH₂OC(O)—, CH₂O—, —CH₂NR⁷C(O)— or —CH₂OC(O)NR⁷—;
L₂ is —OC(O)—, —O—, —NR⁷C(O)— or —OC(O)NR⁷—;
R⁴ is -L₃R⁵ or -L₄R⁵;
R⁵ is N(R⁷)₂, —OR⁷, —P(O)(OR⁷)₂, —C(O)OR⁷, —NR⁷C(O)L₃R⁸, —NR⁷C(O)L₄R⁸, —OL₃R⁶, —C(O)NR⁷L₃R⁸, —C(O)NR⁷L₄R⁸, —S(O)₂OR⁷, —OS(O)₂OR⁷, C₁-C₆alkyl, a C₆aryl, a C₁₀aryl, a C₁₀aryl, 5 to 14 ring membered heteroaryl containing 1 to 3 heteroatoms selected from O, S and N, C₃-C₈cycloalkyl or a 5 to 6 ring membered heterocycloalkyl containing 1 to 3 heteroatoms selected from O, S and N, wherein the aryl, heteroaryl, cycloalkyl and heterocycloalkyl of R⁵ are each unsubstituted or the aryl, heteroaryl, cycloalkyl and heterocycloalkyl of R⁵ are each substituted with 1 to 3 substituents independently selected from —OR⁹, -OL₃R⁶, -OL₄R⁶, —OR⁷, and —C(O)OR⁷;
L₃ is a C₁-C₁₀alkylene, wherein the C₁-C₁₀alkylene of L₃ is unsubstituted, or the C₁-C₁₀alkylene of L₃ is substituted with 1 to 4 R⁶ groups, or the C₁-C₁₀alkylene of L₃ is substituted with 2 C₁-C₆alkyl groups on the same carbon atom which together, along with the carbon atom they are attached to, form a C₃-C₈cycloakyl;
L₄ is —((CR⁷R⁷)ₚO)_{q}(CR¹⁰R¹⁰)ₚ— or —(CR¹¹R¹¹)((CR⁷R⁷)ₚO)_{q}(CR¹⁰R¹⁰)ₚ—, wherein each R¹¹ is a C₁-C₆alkyl groups which together, along with the carbon atom they are attached to, form a C₃-C₈cycloakyl;
each R⁶ is independently selected from halo, C₁-C₆alkyl, C₁-C₆alkyl substituted with 1-2 hydroxyl groups, —OR⁷, —N(R⁷)₂, —C(O)OH, —C(O)N(R⁷)₂, —P(O)(OR⁷)₂, a C₆aryl, a C₁₀aryl and a C₁₄aryl;
each R² is independently selected from H and C₁-C₆alkyl;
R⁸ is selected from —SR⁷, C(O)OH, P(O)(OR⁷)₂, and a 5 to 6 ring membered heterocycloalkyl containing 1 to 3 heteroatoms selected from O and N;
R⁹ is phenyl;
each R¹⁰ is independently selected from H and halo;
each p is independently selected from 1, 2, 3, 4, 5 and 6, and
q is 1, 2, 3 or 4;
and where formula (K) is: 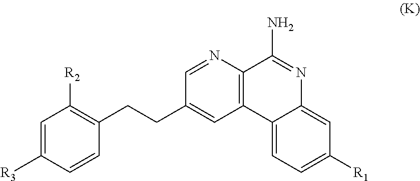
wherein:
R¹ is H, C₁-C₆alkyl, —C(R⁵)₂OH, -L¹R⁵, -L¹R⁶, -L²R⁵, -L²R⁶, —OL²R⁵, or —OL²R⁶;
L¹ is —C(O)— or —O—;
L² is C₁-C₆alkylene, C₂-C₆alkenylene, arylene, heteroarylene or —((CR⁴R⁴)ₚO)_{q}(CH₂)ₚ—, wherein the C₁-C₆alkylene and C₂-C₆alkenylene of L² are optionally substituted with 1 to 4 fluoro groups;
each L³ is independently selected from C₁-C₆alkylene and —((CR⁴R⁴)ₚO)₁(CH₂)ₚ—, wherein the C₁-C₆alkylene of L³ is optionally substituted with 1 to 4 fluoro groups;
L⁴ is arylene or heteroarylene;
R² is H or C₁-C₆alkyl;
R³ is selected from C₁-C₄alkyl, -L³R⁵, -L¹R⁵, -L³R⁷, -L³L⁴L³R⁷, -L³L⁴R⁵, -L³L⁴L³R⁵, —OL³R⁵, —OL³R², —OL³L⁴R², —OL³L⁴L³R⁷, —OR⁸, —OL³L⁴R⁵, —OL³L⁴L³R⁵ and —C(R⁵)₂OH;
each R⁴ is independently selected from H and fluoro;
R⁵ is —P(O)(OR⁹)₂,
R⁶ is —CF₂P(O)(OR⁹)₂ or —C(O)OR¹⁰;
R⁷ is —CF₂P(O)(OR⁹)₂ or —C(O)OR¹⁰;
R⁸ is H or C₁-C₄alkyl;
each R⁹ is independently selected from H and C₁-C₆alkyl;
R¹⁰ is H or C₁-C₄alkyl;
each p is independently selected from 1, 2, 3, 4, 5 and 6, and
q is 1, 2, 3 or 4.

22. The composition of claim 1, wherein the TLR agonist is a TLR7 agonist selected from one of compounds 1 to 102 as defined in WO2012/031140, or a pharmaceutically acceptable salt thereof.

23. (canceled)

24. (canceled)

25. A method of raising an immune response in a subject, comprising the step of administering to the subject the composition of claim 1.

26. A process for preparing the immunogenic composition of claim 1, wherein the process comprises mixing an insoluble metal salt, a TLR agonist, and a rabies virus immunogen.

27. (canceled)

28. (canceled)

29. (canceled)

30. A process for preparing a sterile immunogenic composition, comprising steps of combining (i) a rabies virus immunogen with (ii) a sterile complex of a TLR agonist and an insoluble metal salt.

31. (canceled)

32. A composition comprising: (a) an adjuvant complex comprising a first TLR agonist adsorbed to an insoluble metal salt; (b) an adjuvant complex comprising a second TLR agonist adsorbed to an insoluble metal salt; and (c) a rabies virus immunogen.
